SEQUENCE LISTING (5 sequences)

SEQ ID NO: 1 - protein, 4 aa, Homo sapiens
DAHK

SEQ ID NO: 2 - protein, 8 aa, Artificial Sequence
  metal
DAHGGHAX

SEQ ID NO: 3 - protein, 7 aa, Homo sapiens
  MISC_FEATURE (1)..(7): Xaa = any amino acid
GMXCXXC

SEQ ID NO: 4 - protein, 7 aa, Homo sapiens
  MISC_FEATURE (1)..(7): Xaa = any amino acid
GMTCXXC

SEQ ID NO: 5 - protein, 7 aa, Enterococcus hirae
GMTCANC

(19) United States
(12) Patent Application Publication
(10) Pub. No.: US 2003/0055003 A1
(43) Pub. Date: Mar. 20, 2003

(54) Use of copper chelators to inhibit the inactivation of protein C

(76) Inventors: David Bar-Or, Englewood, CO (US); Richard L. Yukl, Denver, CO (US)

Correspondence Address: SHERIDAN ROSS PC, 1560 BROADWAY, SUITE 1200, DENVER, CO, 80202

(21) Appl. No.: 10/199,285

(22) Filed: Jul. 19, 2002

Related U.S. Application Data
(60) Provisional application No. 60/307,005, filed on Jul. 19, 2001.
(60) Provisional application No. 60/344,514, filed on Dec. 28, 2001.

(51) Int. Cl. A61K 38/00; C07K 5/00; C07K 7/00; A61K 38/06; A61K 38/04; C07K 17/00; C07K 16/00

(52) U.S. Cl. 514/18; 530/330; 530/331

(57) ABSTRACT

The present invention is based on the unexpected discovery that activated protein C (APC) is inactivated by copper. Accordingly, the invention provides improved methods of treating diseases and conditions treatable with APC which utilize a copper chelator to inhibit the inactivation of APC by copper.

This application claims benefit of provisional application No. 60/307,005, filed Jul. 19, 2001, and of provisional application No. 60/344,514, filed Dec. 28, 2001, and the complete disclosures of both of these provisional applications are incorporated herein by reference.

### FIELD OF THE INVENTION

The present invention relates to improved methods of treating diseases and conditions treatable with activated protein C (APC). In particular, the invention relates to methods of treating diseases and conditions treatable with APC which utilize a copper chelator to inhibit the inactivation of APC by copper.

### BACKGROUND

Recombinant human activated protein C (APC) was recently approved for the treatment of severe sepsis (Xigris™, Eli Lilly, Indianapolis, Ind.), and APC may be useful in the treatment of coagulation disorders and severe inflammation. Bernard et al., Crit. Care Med. 29, 2051-2059 (2001); Bernard et al., N. Engl. J. Med. 344, 699-709 (2001); Mizutani et al., Blood 95, 3781-3787 (2000); Taylor et al., J. Clin. Invest. 79, 918-925 (1987); Shibata et al., Circulation 103, 1799-1805 (2001). APC is believed to prevent microvascular thrombosis under physiological conditions and to help control pro-coagulant and pro-inflammatory reactions in diseases such as sepsis. Esmon, Crit. Care Med. 29, S48-51 (2001); Hack et al., Crit. Care Med. 29, S21-27 (2001). The mechanism of action of APC during sepsis is not completely understood, although it appears to be a combination of both anticoagulant and anti-inflammatory activities. Esmon, Crit. Care Med. 29, S48-51 (2001).

APC is a serine protease that functions as an anticoagulant by binding to protein S and proteolytically inactivating factors Va and VIIIa and by stimulating fibrinolysis through neutralization of a plasminogen activator inhibitor. Walker et al., FASEB J. 6, 2561-2567 (1992); Esmon, Arterioscler. Thromb. 12, 135-145 (1992); van Hinsbergh et al., Blood 65, 444-451 (1985). Precursor protein C is produced primarily in the liver. Activation is achieved by the removal of a dodecapeptide at the N-terminus of the heavy chain of protein C. The protein C pathway is initiated when thrombin binds to the endothelial cell surface protein, thrombomodulin, and protein C binds to the endothelial cell protein C receptor. By inactivating factors Va and VIIIa, APC limits the amount of thrombin formed. Esmon, Arterioscler. Thromb. 12, 135-145 (1992).

APC anticoagulant activity can fluctuate in both acute and chronic disease. Some reports suggest that inhibition of APC activity may be pathogenically associated with diseases such as septic shock, disseminated intravascular coagulation (DIC), multiple organ dysfunction syndrome, and atherosclerosis. Fourrier et al., Chest 101, 816-823 (1992); Hoogendoom et al. Blood 78, 2283-2290 (1991); Marshall, Crit. Care Med. 29, S99-106 (2001); Mezzano et al., Br. J. Haematol. 113, 905-910 (2001). Decreased plasma APC activity and beneficial treatment with APC have been associated with critical diseases such as septic shock, purpura fulminans, deep vein thrombophlebitis, DIC, and multiple organ dysfunction syndrome. Esmon, Crit. Care Med. 29, S48-51 (2001); Fourrier et al., Chest 101, 816-823 (1992); Marshall, Crit. Care Med. 29, S99-106 (2001). Several APC inhibitors have been identified, including a heparin-dependent, plasma serine protease protein C inhibitor (serpin), antithrombin III, alpha 1-antitrypsin, and alpha 2-macroglobulin. Esmon, Arterioscler. Thromb. 12, 135-145 (1992); Hoogendoom et al. Blood 78, 2283-2290 (1991); Espana et al., Blood 77, 1754-1760 (1991); Hermans et al., Biochem. J. 295, 239-245 (1993); Espana et al., Thromb. Res. 59, 593-608 (1990); Watanabe et al., Am. J Hematol. 65, 35-40 (2000).

As far as is known, there are no prior reports of copper inhibiting APC anticoagulant activity. Copper is normally bound to plasma carrier proteins such as ceruloplasmin, albumin, and macroglobulins in an equilibrium of both non-specific (exchangeable) and tight (non-exchangeable) binding sites. Linder et al., Biochemistry of Copper (Plenum Press, New York, 1991). However, critical illnesses like sepsis often cause generalized or localized ischemia and acidosis, which can release copper ions. Mizock et al., Crit. Care Med. 20, 80-93 (1992); Pastores et al., Am. J Gastroenterol. 91, 1697-1710 (1996); Machiedo et al., Arch. Surg. 123, 424-427 (1988); Berenshtein et al., J. Mol. Cell. Cardiol. 29, 3025-3034 (1997); Lamb et al., FEBS Lett. 338, 122-126 (1994); Srinivas et al., Scand. J. Clin. Lab. Invest. 48, 495-500 (1988); Sussman et al., Methods Enzymol. 186, 711-723 (1990); Halliwell et al., Methods Enzymol. 186, 1-85 (1990). Free copper released by ischemia and acidosis during sepsis would be available to bind to endogenous or therapeutically administered APC.

### SUMMARY OF THE INVENTION

The present invention is based on the unexpected discovery that activated protein C (APC) is inactivated by copper. Accordingly, the invention provides improved methods of treating diseases and conditions treatable with APC which utilize a copper chelator to inhibit the inactivation of APC by copper.

One method of the invention comprises administering to an animal in need of treatment with APC an effective amount of a copper chelator to inhibit the inactivation of APC by copper. An effective amount of one of the following is also administered to the animal:

(a) APC;

(b) protein C, an agent that increases the synthesis of protein C in the animal, or both;

(c) an activator of protein C; or

(d) a combination of one or more of (a), (b) and (c).

The protein C, the agent that increases the synthesis of protein C, and/or the activator of protein C are administered to the animal to increase the in vivo production of APC from protein C (endogenously produced protein C and/or protein C administered to the animal).

A second method of the invention comprises contacting an effective amount of a copper chelator with a composition comprising one of the following:

(a) APC;

(b) protein C, an agent that increases the synthesis of protein C in the animal, or both;

(c) an activator of protein C; or

(d) a combination of one or more of (a), (b) and (c);

so as to bind any copper present in the composition. Then, an effective amount of the APC, protein C, the agent that increases the synthesis of protein C, the activator of protein C, or the combination of one or more of them is administered to an animal in need of treatment with APC.

### BRIEF DESCRIPTION OF THE DRAWINGS

FIGS. 1A-D: Formulas of tetrapeptide Asp Ala His Lys [SEQ ID NO:1] showing points of possible substitution.

FIGS. 2A-B: Schematic diagrams of the synthesis of derivatives of the tetrapeptide Asp Ala His Lys [SEQ ID NO:1] coming within the formula of FIG. 1C (FIG. 2A) and FIG. 1B (FIG. 2B).

FIG. 3A-B: Formulas of cyclohexane diamine derivatives.

FIGS. 3C-D: Schematic diagrams of syntheses of cyclohexane diamine derivatives of the tetrapeptide Asp Ala His Lys [SEQ ID NO:1].

FIG. 4: Formula of a tetraacetic acid derivative of the tetrapeptide Asp Ala His Lys [SEQ ID NO:1].

FIG. 5: Formula of a bispyridylethylamine derivative of the tetrapeptide Asp Ala His Lys [SEQ ID NO:1].

FIGS. 6A-B: Formulas of mesoporphyrin IX with (FIG. 6B) and without (FIG. 6A) a bound metal ion M.

FIG. 6C: Formula of mesoporphyrin IX derivative of the tetrapeptide Asp Ala His Lys [SEQ ID NO:1].

FIG. 7: Formulas of monosaccharides.

FIG. 8A: Formulas of peptide dimers according to the invention.

FIGS. 8B-C: Diagrams illustrating the synthesis of peptide dimers according to the invention.

FIG. 9. Graph showing effects of copper (Cu) alone, human serum albumin (HSA) alone, and various ratios of HSA:Cu on activated protein C (APC) anticoagulant activity. The data are expressed as percent change from baseline APC activity (n=3 for each bar, mean±standard deviation).

FIG. 10. Graph showing effects of copper (Cu) alone, the tetrapeptide D-Asp D-Ala D-His D-Lys (d-DAHK) alone, and various ratios of d-DAHK:Cu on APC anticoagulant activity. The data are expressed as percent change from baseline APC activity (n=3 for each bar, mean±standard deviation).

### DETAILED DESCRIPTION OF THE PRESENTLY-PREFERRED EMBODIMENTS

Activated protein C (APC) has been reported to be effective in the treatment of the following diseases and conditions:

(a) an acquired hypercoagulable state or an acquired protein C deficiency associated with sepsis, septic shock, purpura fulminans, meningococcal sepsis, bone marrow or other transplantations, severe bums, pregnancy, major surgery, severe trauma, or adult respiratory distress syndrome (ARDS) (see, e.g., U.S. Pat. Nos. 6,156,734 and 6,268,344 and PCT application WO 99/20293);

(b) diseases or conditions involving intravascular coagulation, such as deep vein thrombosis, pulmonary embolism, peripheral arterial thrombosis, emboli originating from the heart or peripheral arteries, acute myocardial infarction, thrombotic strokes and disseminated intravascular coagulation (DIC) (see, e.g., U.S. Pat. No. 5,151,268);

(c) metastasic cancers and invasive cancers (see U.S. Pat. No. 5,151,268);

(d) diseases or conditions associated with apoptosis, such as Alzheimer's disease, Parkinson's disease, autoimmune diseases, viral infections, rheumatoid arthritis, inflammatory bowel disease, vasculitis, ischemic renal failure, insulin-dependent diabetes mellitus, pancreatitis, psoriasis, multiple sclerosis, Hashimoto's thyroiditis, Graves disease, systemic lupus erythematosus, autoimmune gastritis, fibrosing lung disease, HIV-induced lymphoma, fulminant viral hepatitis B, fulminant viral hepatitis C, chronic hepatitis, chronic cirrhosis, H. pyrlori-associated ulceration, atherosclerosis, cytoprotection during cancer treatment, chronic glomerulonephritis, osteoporosis, aplastic anemia, and myelodysplasia (see, e.g., PCT application WO 01/72328);

(e) a disease or condition induced by nuclear factor kappa B (NF-KB), such as neuronal degeneration diseases, graft versus host reactions, acute inflammatory conditions, systemic inflammatory responses, acute phase response, ischemic reperfusion injury, atherosclerosis, HIV infections, and cancer (see, e.g., PCT application WO 01/72328);

(f) a disease or condition where TNF-A is a primary modulator of pathophysiology, such as Crohn's disease, ulcerative colitis, arthritis, acute peritoneal inflammation, and heart failure (see, e.g., PCT application WO 01/72328);

(g) a disease or condition in which major histocompatibility complex (MHC) class 1 or HLA-B null allele is a modulator of immune function, such as organ transplantation, infectious diseases and autoimmune diseases (see, e.g., PCT application WO 01/72328);

(h) a disease or condition where proliferating cell nuclear antigen (PCNA) or Gu protein is a regulator of cell growth and survival, such as cell growth of endothelial cells and angiogenesis (see, e.g., PCT application WO 01/72328);

(i) a disease or condition due to endothelial cell activation and platelet adhesion, such as coronary artery atherosclerosis, arterial restenosis following balloon angioplasty, hypertension, cardiac failure, coronary disease after transplantation, and pregnancy-induced hypertension and pre-eclampsia (see, e.g., PCT application WO 01/72328);

(j) a disease or condition where cell-cell adhesion is a modulator of pathophysiology (see, e.g., PCT application WO 01/72328);

(k) diseases or conditions involving inflammation and neuropathological disorders, such as ischemia, ischemia reperfusion, Alzheimer's disease, Huntington disease or chorea, hypoxia, cell death due to epilepsy, amyotrophic lateral sclerosis, multiple sclerosis, mental retardation, neurodegenerative changes resulting from aging, inflammatory bowel diseases (e.g., Crohn's disease and ulcerative colitis), shock, glomerulonephritis, coronary arterial occlusion, cardiac arrhythmias, congestive heart failure, cardiomyopathy, bronchitis, acute allergic reactions and hypersensitivity, trauma, graft/transplant rejection, myocarditis, insulin dependent diabetes, arthritis, chronic inflammatory conditions of the skin, and ARDS (see, e.g., PCT applications WO 01/56532 and WO 01/72328);

(l) diseases or conditions where anti-calreticulin antibodies are a modulator of pathophysiology, such as systemic lupus erythematosus, Sjogren's syndrome, onchocerciasis, rheumatoid arthritis, mixed connective tissue disease and complete congenital heart block (see, e.g., PCT application WO 01/72328);

(m) diseases or conditions associated with elevated levels of thrombospondin (TSP-1) and TGF-β, such as breast cancer, gastrointestinal malignancies, gynecological cancers, lung cancer, kidney fibrosis, and cardiac hypertrophy following myocardial infarction (see, e.g., PCT application WO 01/72328); and

(n) diseases or conditions associated with elevated levels of RDC1, such as bacterial, fungal, protozoan and viral infections, pain, cancers, anorexia, bulimia, asthma, Parkinson's disease, acute heart failure, hypotension, hypertension, urinary retention, osteoporosis, angina pectoris, ulcers, allergies, benign prostatic hypertrophy, and psychotic and neurological disorders (e.g., anxiety, schizophrenia, manic depression, delirium, dementia, severe mental retardation and dyskinesias such as Huntington's disease or Gilles de la Tourett's syndrome) (see, e.g., PCT application WO 01/72328).

Methods of making APC, suitable pharmaceutical compositions containing APC, and effective doses and schedules for administration of APC for these diseases and conditions are known. See, e.g., U.S. Pat. Nos. 6,395,270, 6,268,344, 6,162,629, 6,159,468, 6,156,734, 5,831,025, 5,330,907, 5,151,268, and 4,981,952, PCT applications WO 89/12685, WO 98/48818, WO 99/20293, WO 01/56532, WO 01/59084, and WO 01/72328, and EP patent no. 726,076, the complete disclosures of all of which are incorporated herein by reference.

In particular, APC may be prepared by in vitro activation of protein C purified from plasma or prepared by recombinant DNA techniques by methods well known in the art. See, e.g., U.S. Pat. Nos. 4,981,952, 5,151,268, 5,831,025, 6,156,734, 6,268,344, and 6,395,270. Alternatively, APC may be prepared directly by recombinant DNA techniques. See, e.g., U.S. Pat. Nos. 4,981,952, 5,151,268, 6,156,734, 6,268,344 and 6,395,270. APC may be from any species of animal, but human APC is preferred. Fragments and derivatives of APC can also be used in the practice of the invention, provided that they exhibit the activities described herein. See, e.g., U.S. Pat. Nos. 5,151,268, 5,453,373 and 5,516,650 and PCT applications WO 89/12685, WO 01/56532, WO 01/59084, and WO 01/72328.

Suitable pharmaceutical compositions of APC comprise the APC and a pharmaceutically-acceptable carrier. See, e.g., U.S. Pat. Nos. 6,395,270 and 6,159,468 and PCT applications WO 98/48818, WO 01/56532 and WO 01/72328. A preferred composition is one that is a stable lyophilized product of high purity comprising a bulking agent (such as sucrose, mannitol, trehalose, and raffinose), a salt (such as sodium chloride and potassium chloride), a buffer (such as sodium citrate, Tris-acetate, and sodium phosphate), and APC. A preferred stable lyophilized composition will comprise a weight ratio of about 1 part APC, between about 7-8 parts salt, and between about 5-7 parts bulking agent. An example of such a stable lyophilized composition is: 5.0 mg APC, 30 mg sucrose, 38 mg NaCl, and 7.56 mg citrate, pH 6.0, per vial.

APC is preferably administered parenterally (preferably intravenously), most preferably by continuous intravenous infusion. See, e.g., U.S. Pat. No. 6,268,344 and PCT application WO 01/72328. Preferably, from about 0.01 μg/kg/hr to about 50 μg/kg/hr of APC, more preferably from about 1 μg/kg/hr to about 40 μg/kg/hr, even more preferably from about 10 μg/kg/hr to about 30 μg/kg/hr, most preferably about 24 μg/kg/hr, are administered to a human patient by continuous infusion for a period of from about 1 hour to about 240 hours, more preferably for a period of from about 1 hour to about 144 hours, most preferably from about 24 hours to about 96 hours. APC may also be administered by injecting a dose of from about 0.01 mg/kg/day to about 10 mg/kg/day, B.I.D. (2 times a day), for one to ten days, most preferably for three days. As another alternative, APC can be administered by injecting a portion (⅓ to ½) of the appropriate dose per hour as a bolus injection over a time of from about 5 minutes to about 120 minutes, followed by continuous infusion of the appropriate dose for up to 240 hours. The preferred plasma levels obtained from the amount of APC administered will be from about 0.02 ng/ml to about 500 ng/ml, more preferably from about 2 ng/ml to about 200 ng/ml, most preferably from about 35 ng/ml to about 65 ng/ml.

In other alternatives, APC can be administered by local delivery through an intracoronary catheter as an adjunct to high-risk angioplasty (with and without stenting and with or without combination antithrombotic therapy with or without anti-platelet agents). The amount of APC administered will be from about 0.01 mg/kg/day to about 10.0 mg/kg/day by continuous infusion, bolus injection, or a combination thereof. In another alternative, APC can be injected directly into joints. In yet another alternative, APC can be administered subcutaneously at a dose of 0.01 mg/kg/day to about 10.0 mg/kg/day to ensure a slower release into the bloodstream. Formulation of subcutaneous preparations will be done using known methods to prepare such pharmaceutical compositions.

A particularly preferred formulation of APC is the product sold by Eli Lilly and Co., Indianapolis, Ind., under the trademark Xigris™. Xigris™ is supplied as a sterile, lyophilized powder for intravenous infusion. The 5 mg vials of Xigris™ contain 5.3 mg/vial of human recombinant APC, 31.8 mg/vial sucrose, 40.3 mg/vial NaCI, and 10.9 mg/vial sodium citrate, and the 20 mg vials of Xigris™ contain 20.8 mg/vial of human recombinant APC, 124.9 mg/vial sucrose, 158.1 mg/vial NaCl, and 42.9 mg/vial sodium citrate. The vials are reconstituted with Sterile Water for Injection, USP, to give a concentration of about 2 mg/ml APC, and this diluted APC is then added to 0.9% Sodium Chloride Injection to give a concentration of from about 100 to about 1000 μg/ml APC for administration to a patient. For severe sepsis, Xigris™ is administered by continuous infusion at a rate of from about 12 μg/kg/hr to about 30 μg/kg/hr to give a steady state plasma concentration of about 45 ng/ml APC after about two hours of infusion.

The diseases and conditions listed above can also be treated by increasing endogenous production of APC. See, e.g., PCT application WO 93/09807. This can be accomplished in a variety of ways. For instance, this can be accomplished by administering an effective amount of protein C which will be activated in vivo by the endogenous protein C pathway to produce APC. See, e.g., U.S. Pat. No. 5,151,268 and PCT application WO 93/09807. As noted above, protein C can be purified from plasma or can be made by recombinant DNA techniques. See, e.g., U.S. Pat. Nos. 4,959,318, 4,981,952, 5,093,117, 5,151,268, 5,571,786, 6,156,734, 6,268,344, and 6,395,270. Suitable pharmaceutical compositions comprising protein C are known (see, e.g., U.S. Pat. Nos. 5,151,268 and 5,571,786). Protein C is preferably administered parenterally, most preferably intravenously, at a dose of from about 1 μg/day to about 500 mg/day or from about 1 IU/kg/day to about 6000 IU/kg/day for a human patient. See, e.g., U.S. Pat. Nos. 5,151,268 and 5,571,786. One IU is that amount of APC amidolytic activity in 1 ml of normal plasma.

Endogenous production of APC can also be increased by administering an amount of an agent that increases the synthesis of protein C in the animal. See, e.g., PCT application WO 93/09807. Suitable agents include anabolic steroids (e.g., danazolol). See, e.g., PCT application WO 93/09807.

In addition, endogenous production of APC can be increased by administering an amount of a protein C activator effective to cause the production of APC in vivo from endogenously synthesized protein C and/or from co-administered protein C. See, e.g., PCT application WO 93/09807. A protein C activator is any compound that causes or increases the generation of APC. Suitable protein C activators include thrombin, a-thrombin, active site acylated thrombin, thrombin analogs and mutants (e.g., thrombin E192Q and thrombin K52E), soluble thrombin-thrombomodulin complexes, agents that would prevent clearance or decay of thrombin-thrombomodulin complexes, agents that enhance the synthesis or delay the clearance of thrombomodulin, a venom (such as Protac or Russel Viper venom), factor Xa, plasmin, trypsin, and any other venom, enzyme or compound capable of causing or increasing the generation of APC from protein C. See, e.g., PCT application WO 93/09807. Preferred protein C activators are thrombin and active site acylated thrombin. The protein C activator is preferably administered parenterally, most preferably intravenously. See, e.g., PCT application WO 93/09807. Preferably, an amount of the protein C activator is administered which increases the blood level of APC 3-5 times over the normal level and/or that gives a blood concentration of APC of from about 10 ng/ml to about 760 ng/ml. See PCT application WO 93/09807. For thrombin, a dosage of from about 0.05 U/kg/min to about 2 U/kg/min is effective to achieve these levels of APC. See PCT application WO 93/09807. For active site acylated thrombin, a dosage is used which will produce (essentially in a “controlled release” manner) from about 0.05 U/kg/min to about 2 U/kg/min of thrombin activity as the active site is deacylated in vivo. See PCT application WO 93/09807. One unit (U) of thrombin is generally known in the art and means equivalent fibrinogen clotting activity to one NIH unit of reference enzyme using the same assay. See PCT application WO 93/09807 and the Fenton et al., Thromb. Res., 4, 809-817 (1974) reference cited therein.

Notwithstanding the foregoing, it is understood by those skilled in the art that the dosage amount of the APC, protein C, agent that increases the synthesis of protein C, and/or protein C activator will vary with the particular compound or combination of compounds employed, the disease or condition to be treated, the severity of the disease or condition, the route(s) of administration, the rate of excretion of the compound, the duration of the treatment, the identify of any other drugs being administered to the animal, the age, size and species of the animal, and like factors known in the medical and veterinary arts. In general, a suitable daily dose of a compound or combination of compounds will be that amount which is the lowest dose effective to produce a therapeutic effect. The dosage amount, dosage form and mode of administration will be determined by an attending physician or veterinarian within the scope of sound medical judgment. Effective dosage amounts, dosage forms, and modes of administration for the various compounds and combination(s) of compounds can be determined empirically and making such determinations is within the skill of the art.

The present invention provides improved methods of treating a disease or condition treatable with APC. The present invention is based on the unexpected discovery that APC is inactivated by copper, and the improved methods of the invention utilize a copper chelator to inhibit the inactivation of APC by copper. As used herein, “inhibit” and variants thereof mean to reduce or prevent the inactivation of APC by copper and/or to wholly or partially reactivate or restore the activity of APC that has been inactivated by copper. As used herein, “inactivate” and variants thereof mean to reduce or completely abolish the activity of APC.

Any copper chelator may be used in the practice of the present invention. As used herein, “copper chelator” means any compound that binds Cu(II) ions.

Preferred copper chelators for use in the practice of the invention include certain albumins which possess an N-terminal copper binding site of high affinity and fragments of these albumins which comprise the N-terminal copper binding site. These albumins include human, rat and bovine serum albumins. Particularly preferred is human serum albumin or a fragment thereof that comprises the high-affinity N-terminal copper-binding sequence Asp Ala His. Methods of preparing albumin and fragments of albumin from plasma/serum and by recombinant DNA techniques are well known in the art. See, e.g., U.S. Pat. Nos. 4,990,447, 5,037,744, 5,250,662, 5,260,202, 5,380,712, 5,440,018, 5,503,993, 5,521,287, 5,707,828, 5,728,553, 5,756,313, 5,759,802, 5,849,874, 5,879,907, 6,034,221, and 6,150,504, PCT applications nos. WO 84/03511, WO 89/02467, WO 96/37515, WO 97/31947, WO 99/65943 and WO 01/72959, and EP 73646, 206733 and 308381.

Additional preferred copper chelators for use in the practice of the invention are peptides of the formula:

P₁-P₂,

wherein:

P₁ is:

Xaa₁ Xaa₂ His: or

Xaa₁ Xaa₂ His Xaa₃;

P₂ is (Xaa₄)ₙ;

Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;

Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;

Xaa₃ is glycine, alanine, valine, lysine, arginine, ornithine, aspartic acid, glutamic acid, asparagine, glutamine or tryptophan;

Xaa₄ is any amino acid; and

n is 0-100;

or a physiologically-acceptable salt thereof.

P₁ is a metal-binding peptide sequence that binds transition metal ions of Groups 1b-7b or 8 of the Periodic Table of elements (including V, Co, Cr, Mo, Mn, Ba, Zn, Hg, Cd, Au, Ag, Co, Fe, Ni, and Cu) and other metal ions (including As, Sb and Pb). In particular, P₁ binds Cu(II), Ni(II), Co(II), and Mn(II) with high affinity, and P₁ is a particularly effective copper chelator for inhibiting the inactivation of APC by copper. In addition, it is known that the binding of metal ions by P₁ inhibits (i.e., reduces or prevents) the production of reactive oxygen species (ROS) and/or the accumulation of ROS caused by these metal ions and/or targets the damage done by ROS that may still be produced by the bound metal ions to the peptide itself. As a result, the damage that can be caused by ROS in the absence of the binding of the metal ions to P₁ is reduced. Accordingly, these peptides will provide added advantages in treating diseases and conditions treatable with APC which also involve the production and/or accumulation of ROS. Such diseases and conditions include angioplasty, ARDS, angiogenic diseases, artherosclerosis, arthritis, asthma, autoimmune diseases, cancer, colitis, Crohn's disease, diabetes, emphysema, head injury and traumatic brain injury, infectious diseases, inflammation and inflammatory diseases, metastasis, ischemia, neoplastic diseases, neurological diseases, neurological trauma, neurodegenerative diseases (e.g., Alzheimer's disease, amyotropic lateral sclerosis, Huntington's chorea, Parkinson's disease, multiple sclerosis, and senile dementia), pancreatitis, peripheral vascular disease, pulmonary embolism, renal diseases, reperfusion, sepsis, shock, surgery, transplantation, trauma, vasculitis, and many others (see, e.g., U.S. patent applications Ser. No. ______, filed Jun. 27, 2002, Ser. No. 10/076,071, filed Feb. 13, 2002, and Ser. No. 09/678,202, filed Sep. 29, 2000, and PCT applications PCT/US00/26952, filed Sep. 30, 2000 (published as WO 01/25265), and PCT/US02/04275, filed Feb. 13, 2002, the complete disclosures of which are incorporated herein by reference).

In P₁, Xaa₁ is most preferably Asp, Xaa₂ is most preferably Ala, and Xaa₃ is most preferably Lys (see above). Thus, the preferred sequences of P₁ are Asp Ala His and Asp Ala His Lys [SEQ ID NO: 1]. Most preferably the sequence of P₁ is Asp Ala His Lys [SEQ ID NO: 1]. Asp Ala His is the minimum sequence of the N-terminal metal-binding site of human serum albumin necessary for the high-affinity binding of Cu(lI) and Ni(II), and Lys has been reported to contribute to the binding of these metal ions to this site. Also, Asp Ala His Lys [SEQ ID NO:1] has been found by mass spectometry to bind Fe(II) and to pass through a model of the blood brain barrier. Other preferred sequences for P, include Thr Leu His (the N-terminal sequence of human a-fetoprotein), Arg Thr His (the N-terminal sequence of human sperm protamin HP₂) and HMS HMS His (a synthetic peptide reported to form extremely stable complexes with copper; see Mlynarz et al., Speciation 98: Abstracts, Apr. 21, 1998, http://www.jate.uszeged.hu/spec98/abstr/mlynar.html).

P₂ is (Xaa₄)ₙ, wherein Xaa₄ is any amino acid and n is 0-100. When n is large (n>about 20), the peptides will be effective extracellularly. Smaller peptides are better able to enter cells, and smaller peptides can, therefore, be effective both intracellularly and extracellularly. Smaller peptides are also less subject to proteolysis. Therefore, in P₂, preferably n is 0-10, more preferably n is 0-5, and most preferably n is 0. Although P₂ may have any sequence, P₂ preferably comprises a sequence which (1) binds a transition metal, (2) enhances the ability of the peptide to penetrate cell membranes and/or reach target tissues (e.g., to be able to cross the blood brain barrier), or (3) otherwise stabilizes or enhances the performance of the peptide. P₂ together with P₁ may also be the N-terminal sequence of a protein having an N-terminal metal-binding site with high affinity for copper and nickel, such as human, rat or bovine serum albumin. In the case where n=100, the peptide would have the sequence of approximately domain 1 of these albumins.

The sequences of many peptides which comprise a binding site for transition metal ions are known. See, e.g., U.S. Pat. Nos. 4,022,888, 4,461,724, 4,665,054, 4,760,051, 4,767,753, 4,810,693, 4,877,770, 5,023,237, 5,059,588, 5,102,990, 5,118,665, 5,120,831, 5,135,913, 5,145,838, 5,164,367, 5,591,711, 5,177,061, 5,214,032, 5,252,559, 5,348,943, 5,443,816, 5,538,945, 5,550,183, 5,591,711, 5,690,905, 5,759,515, 5,861,139, 5,891,418, 5,928,955, and 6,017,888, PCT applications WO 94/26295, WO 99/57262 and WO 99/67284, European Patent application 327263, Lappin et al., Inorg. Chem., 17, 1630-34 (1978), Bossu et al., Inorg. Chem., 17, 1634-40 (1978), Chakrabarti, Protein Eng., 4, 57-63 (1990), Adman, Advances In Protein Chemistry, 42, 145-97 (1991), Cotelle et al., J. Inorg. Biochem., 46, 7-15 (1992), Canters et al., FEBS, 325, 39-48 (1993), Regan, Annu. Rev. Biophys. Biomol. Struct., 22, 257-281 (1993), Ueda et al., J. Inorg. Biochem., 55, 123-30 (1994), Ueda et al., Free Radical Biol. Med., 18, 929-33 (1995), Regan, TIBS, 20, 280-85 (1995), Ueda et al., Chem. Pharm. Bull., 43, 359-61 (1995), Bal et al., Chem. Res. Toxicol., 10, 906-914 (1997), Bal et al., Chem. Res. Toxicol., 10; 915-21 (1997), Koch et al., Chem. Biol., 4, 549-60 (1997), Kowalik-Jankowska et al., J. Inorg. Biochem., 66, 193-96 (1997), Harford and Sarkar, Acc. Chem. Res., 30, 123-130 (1997), Prince et al., TIBS, 23, 197-98 (1998), Mlynarz, et al., Speciation 98: Abstracts, http://www.jate.u-szeged.hu/˜spec98/abstr/mlynar.html, and Aitken, Molec. Biotechnol., 12, 241-53 (1999), Whittal et al., Protein Science, 9, 332-343 (2000). P₂ may comprise the sequence of one or more of the metal-binding sites of these peptides.

Preferably, P₂ will comprise the sequence a copper-binding site and/or of an iron-binding site so that P₁-P₂ will be better able to inhibit the inactivation of APC by copper and/or to inhibit the formation and/or accumulation of ROS. The sequences of many peptides which comprise a copper-binding site are known. See, e.g., U.S. Pat. Nos. 4,022,888, 4,461,724, 4,665,054, 4,760,051, 4,767,753, 4,810,693, 4,877,770, 5,023,237, 5,059,588, 5,102,990, 5,118,665, 5,120,831, 5,135,913, 5,145,838, 5,164,367, 5,177,061, 5,214,032, 5,252,559, 5,348,943, 5,443,816, 5,538,945, 5,550,183, 5,591,711, 5,690,905, 5,759,515, 5,861,139, 5,891,418, 5,928,955, and 6,017,888, PCT applications WO 94/26295, WO 99/57262, WO 99/67284 and WO 00/36136, European Patent application 327263, Lappin et al., Inorg. Chem., 17, 1630-34 (1978), Chakrabarti, Protein Eng., 4, 57-63 (1990), Adman, Advances In Protein Chemistry, 42, 145-97 (1991), Canters et al., FEBS, 325, 39-48 (1993), Regan, Annu. Rev. Biophys. Biomol. Struct., 22, 257-281 (1993), Ueda et al., J. Inorg. Biochem., 55, 123-30 (1994), Ueda et al., Free Radical Biol. Med., 18, 929-33 (1995), Regan, TIBS, 20, 280-85 (1995), Ueda et al., Chem. Pharm. Bull., 43, 359-61 (1995), Bal et al., Chem. Res. Toxicol., 10, 906-914 (1997), Bal et al., Chem. Res. Toxicol., 10, 915-21 (1997), Koch et al., Chem. Biol., 4, 549-60 (1997), Kowalik-Jankowska et al., J. Inorg. Biochem., 66, 193-96 (1997), Harford and Sarkar, Acc. Chem. Res., 30, 123-130 (1997), Prince et al., TIBS, 23, 197-98 (1998), Mlynarz, et al., Speciation 98: Abstracts, http://www.jate.u-szeged.hu/˜spec98/abstr/mlynar.html, and Aitken, Molec. Biotechnol., 12, 241-53 (1999), Whittal et al., Protein Science, 9, 332-343 (2000). The sequences of peptides which comprise an iron-binding site are known. See, e.g., U.S. Pat. Nos. 4,022,888, 4,461,724, 5,102,990, 5,120,831, 5,252,559, 5,443,816, 5,550,183, 5,690,905, 5,759,515, 5,891,418, 5,928,955, PCT applications WO 99/57262, WO 99/67284 and WO 00/36136, European Patent application 327263, Lappin et al., Inorg. Chem., 17, 1630-34 (1978), Chakrabarti, Protein Eng., 4, 57-63 (1990), Adman, Advances In Protein Chemistry, 42, 145-97 (1991), Prince et al., TIBS, 23, 197-98 (1998), and Aitken, Molec. Biotechnol., 12, 241-53 (1999), Whittal et al., Protein Science, 9, 332-343 (2000).

When P₂ comprises a metal-binding site, it preferably has a sequence which includes a short spacer sequence between P₁ and the metal binding site of P₂, so that the metal-binding sites of P₁ and P₂ may potentially cooperatively bind metal ions (similar to a 2:1 peptide:metal complex which gives tighter binding than a 1:1 complex). Preferably, the spacer sequence is composed of 1-5, preferably 1-3, neutral amino acids. Thus, the spacer sequence may be Gly, Gly Gly, Gly Ala Gly, Pro, Gly Pro Gly, etc.

When P₂ comprises a metal-binding site, it preferably has a sequence which includes a short spacer sequence between P₁ and the metal binding site of P₂, so that the metal-binding sites of P₁ and P₂ may potentially cooperatively bind metal ions (similar to a 2:1 peptide:metal complex which gives tighter binding than a 1:1 complex). Preferably, the spacer sequence is composed of 1-5, preferably 1-3, neutral amino acids. Thus, the spacer sequence may be Gly, Gly Gly, Gly Ala Gly, Pro, Gly Pro Gly, etc.

In particular, when P₂ comprises a metal-binding site, it preferably comprises one of the following sequences: (Xaa₄)ₘ Xaa₅ Xaa₂ His Xaa₃ or (Xaa₄)ₘ Xaa₅ Xaa₂ His. Xaa₂, Xaa₃ and Xaa₄ are defined above, and m is 0-5, preferably 1-3. When P₂ comprises one of these sequences, it can bind copper. The Xaa₄ amino acid(s), if present, form(s) a short spacer sequence between P₁ and the metal-binding site of P₂ so that the metal-binding sites of P₁ and P₂ may cooperatively bind copper and other metals, and Xaa₄ is preferably a neutral amino acid (see the previous paragraph). Xaa₅ is an amino acid which comprises a 6-amino group (preferably Orn or Lys, more preferably Orn) having the Xaa₄ amino acid(s), if present, or PI attached to it by means of the δ-amino group. See Harford and Sarkar, Acc. Chem. Res., 30, 123-130 (1997) and Shullenberger et al., J. Am. Chem. Soc., 115, 11038-11039 (1993) (as a result of this means of attachment, the α-amino group of Xaa₅ can still participate in binding copper and nickel by means of the ATCUN motif). Thus, for instance, P₁-P₂ could be Asp Ala His Gly Gly (δ)-Orn Ala His [SEQ ID NO:2].

In addition, P₂ may comprise one of the following sequences: [(Xaa₄)ₘ Xaa₅ Xaa₂ His Xaa₃]ᵣ, [(Xaa₄)ₘ Xaa₁ Xaa₂ His]ᵣ, [(Xaa₄)ₘ Xaa₅ Xaa₂His Xaa₃ (Xaa₄)ₘ Xaa₅Xaa₂His]ᵣ, and [(Xaa₄)ₘ Xaa₅ Xaa₂ His(Xaa₄)ₘ Xaa₅ Xaa₂ His Xaa₃]ᵣ, wherein Xaa₂, Xaa₃, Xaa₄, Xaa₅and m are defined and described above, and r is 2-100. In this manner metal-binding polymers that can bind copper may be formed.

In another preferred embodiment, P₂ comprises a peptide sequence that can bind Cu(I). Cu(II) is converted to Cu(I) in the presence of ascorbic acid or other reducing agents, and the Cu(I) reacts with oxygen to produce ROS. P₁ can bind Cu(II) tightly (see above) and is very effective by itself in chelating copper and inhibiting the production of ROS. However, it would be desirable to also employ a P₂ which could bind Cu(I).

Peptide sequences which can bind Cu(I) are known in the art. See, e.g, Pickering et al., J. Am. Chem. Soc., 115, 9498-9505 (1993); Winge et al., in Bioinorganic Chemistry Of Copper, pages 110-123 (Karlin and Tyeklar, eds., Chapman & Hall, New York, N.Y., 1993); Koch et al., Chem & Biol., 4, 549-560 (1997); Cobine et al., in Copper Transport And Its Disorders, pages 153-164 (Leone and Mercer eds., Kluwer Academic/Plenum Publishers, New York, N.Y., 1999). These sequences include:

Met Xaa₄ Met,

Met Xaa₄ Xaa₄ Met,

Cys Cys,

Cys Xaa₄ Cys,

Cys Xaa₄ Xaa₄ Cys,

Met Xaa₄ Cys Xaa₄ Xaa₄ Cys,

Gly Met Xaa₄ Cys Xaa₄ Xaa₄ Cys [SEQ ID NO:3],

Gly Met Thr Cys Xaa₄ Xaa₄ Cys [SEQ ID NO:4], and

Gly Met Thr Cys Ala Asn Cys [SEQ ID NO:5],

wherein Xaa₄ is defined above. Glutathione (γ-Glu Cys Gly) is also known to bind Cu(I). Additional Cu(I)-binding peptide sequences can be identified using a metallopeptide combinatorial library as described in, e.g., PCT application WO 00/36136. Preferably, the Cu(I)-binding peptide comprises the sequence Cys Xaa₄ Xaa₄ Cys (e.g., Gly Met Xaa₄ Cys Xaa₄ Xaa₄ Cys [SEQ ID NO:3], more preferably Gly Met Thr Cys Xaa₄ Xaa₄ Cys [SEQ ID NO:4], most preferably Gly Met Thr Cys Ala Asn Cys [SEQ ID NO:5]).

To enhance the ability of the P₁-P₂ peptide to penetrate cell membranes and/or reach target tissues, P₂ is preferably hydrophobic or an arginine oligomer (see Rouhi, Chem. & Eng. News, 49-50 (Jan. 15, 2001)). When P₂ is hydrophobic, it preferably contains 1-3 hydrophobic amino acids (e.g., Gly Gly), preferably D-amino acids. A hydrophobic P₂ may be particularly desirable for uses of P₁-P₂ where P₁-P₂ must cross the blood brain barrier. The arginine oligomer preferably contains 6-9 Arg residues, most preferably 6-9 D-Arg residues (see Rouhi, Chem. & Eng. News, 49-50 (Jan. 15, 2001). The use of a P₂ which is an arginine oligomer may be particularly desirable when P₁-P₂ is to be administered topically or transdermally.

The amino acids of the peptide may be L-amino acids, D-amino acids, or a combination thereof. Preferably, at least one of the amino acids of P₁ is a D-amino acid (preferably Xaa₁ and/or His), except for β-Ala, when present. Most preferably, all of the amino acids of P₁, other than β-Ala, when present, are D-amino acids. Also, preferably about 50% of the amino acids of P₂ are D-amino acids, and most preferably all of the amino acids of P₂ are D-amino acids. D-amino acids are preferred because peptides containing D-amino acids are resistant to proteolytic enzymes, such as those that would be encountered upon administration of the peptide to an animal (including humans). Also, the use of D-amino acids would not alter the ability of the peptide to bind metal ions, including the ability of the peptide to bind copper with high affinity.

The peptides of the invention may be made by methods well known in the art. For instance, the peptides, whether containing L-amino acids, D-amino acids, or a combination of L- and D-amino acids, may be synthesized by standard solid-phase peptide synthesis methods. Suitable techniques are well known in the art, and include those described in Merrifield, in Chem. Polypeptides, pp. 335-61 (Katsoyannis and Panayotis eds. 1973); Merrifield, J. Am. Chem. Soc., 85, 2149 (1963); Davis et al., Biochem. Int'l, 10, 394-414 (1985); Stewart and Young, Solid Phase Peptide Synthesis (1969); U.S. Pat. Nos. 3, 941,763 and 5,786,335; Finn et al., in The Proteins, 3rd ed., vol. 2, pp. 105-253 (1976); and Erickson et al. in The Proteins, 3rd ed., vol. 2, pp. 257-527 (1976). See also, Polish Patent 315474 (synthesis of HMS-containing peptides) and Shullenberger et al., J. Am. Chem. Soc., 115, 1103811039 (1993) (synthesis of (δ)-Orn-containing peptides). Alternatively, the peptides may be synthesized by recombinant DNA techniques if they contain only L-amino acids. Recombinant DNA methods and suitable host cells, vectors and other reagents for use therein, are well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989).

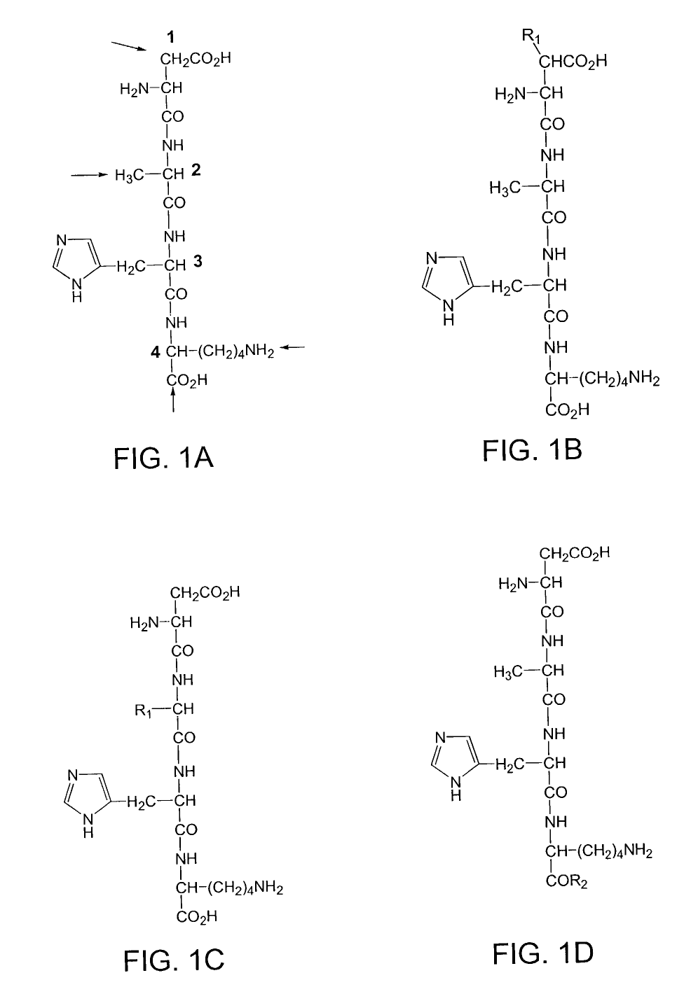

The invention further comprises derivatives of the peptide P₁-P₂, whether composed of L-amino acids, D-amino acids, or a combination of L- and D-amino acids, which are more resistant to proteolytic enzymes, more lipid soluble (to allow the peptides to more readily penetrate cell membranes and/or reach target organs, such as the brain), or both. As illustrated in FIG. 1A, P₁ can be modified in the regions indicated by the arrows without altering the metal binding function of P₁. In particular, P₁ can be substituted at carbons 1 or 2 with R₁, and the terminal —COOH of P₁ can be substituted with protecting group R₂ (FIGS. 1B-D). P₂ can be modified in ways similar to those described for P₁ to make P₂ more resistant to proteolytic enzymes, more lipid soluble, or both.

R₁ can be a straight-chain or branched-chain alkyl containing from 1 to 16 carbon atoms, and the term “alkyl” includes the R and S isomers. R₁ can also be an aryl or heteroaryl containing 1 or 2 rings. The term “aryl” means a compound containing at least one aromatic ring (e.g., phenyl, naphthyl, and diphenyl). The term “heteroaryl” means an aryl wherein at least one of the rings contains one or more atoms of S, N or O. These substitutions do not substantially decrease the ability of P₁ to bind metal ions. In particular, the ability of P₁ to bind copper with high affinity is not decreased by these substitutions. For instance, some of the substituents, such as a n-butyl attached to carbon 2 (see FIG. 1C, R₁ is n-butyl) should increase the affinity of the peptide for metal ions, such as copper, due to the inductive effect of the alkyl group. Substitution of carbon 2 (FIG. 1C) with an aryl, heteroaryl, or a long chain alkyl (about 6-16 carbon atoms) should enhance transport of the peptide across lipid membranes.

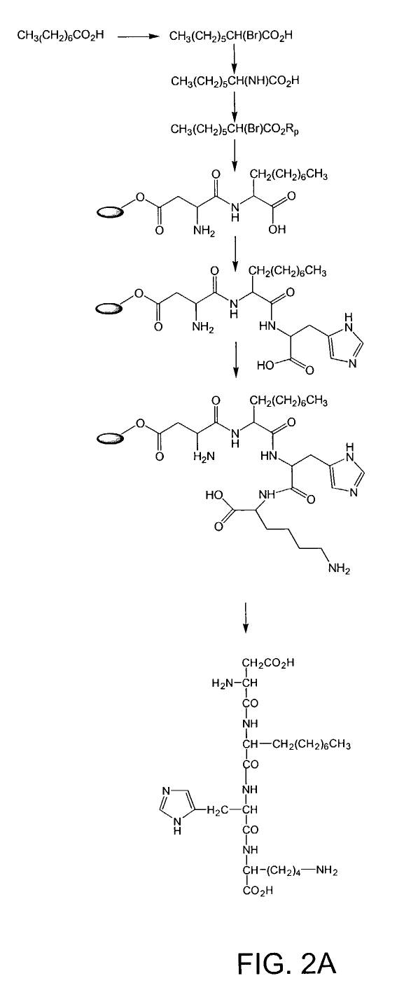

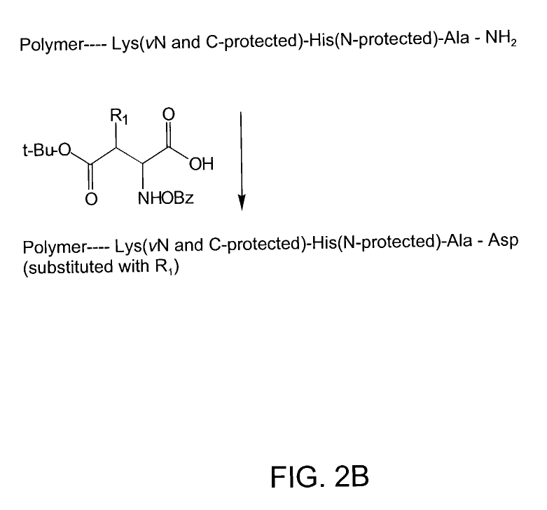

As noted above, methods of synthesizing peptides by solid phase synthesis are well known. These methods can be modified to prepare the derivatives shown in FIGS. 1B-C. For example, the derivative of P₁ illustrated in FIG. 1C, wherein R₁ is octyl, can be prepared as illustrated in FIG. 2A. In FIG. 2A, the elliptical element represents the polymer resin and Rₚ is a standard carboxyl protecting group. As illustrated in FIG. 2A, octanoic acid (freshly distilled) is treated with dry bromine followed by phosphorus trichloride. The mixture is heated to about 100° C. and kept at that temperature for 4 hours. α-Bromooctanoic acid is obtained as a colorless liquid upon distillation. Amination of the bromoacid is achieved by allowing the acid and an ammonia solution to stand at 40-50° C. for 30 hours. The octyl derivative of the amino acid is obtained by removing ammonium bromide with methanol washes. Classical resolution methods give the desired optically-pure D-form. Other derivatives wherein R₁ is an alkyl, aryl or heteroaryl can be prepared in the manner illustrated in FIG. 2A.

In addition, the derivative of P₁ illustrated in FIG. 1B, wherein R₁ is phenyl, can be prepared as illustrated in FIG. 2B. In FIG. 2B, Polymer is the resin, t-Bu is t-butyl, and Bz is benzyl. Other derivatives wherein R₁ is an alkyl, aryl or heteroaryl can be prepared in the manner illustrated in FIG. 2B.

R₂ can be —NH₂, —NHR₁, —N(R₁)₂, —OR₁, or R₁(see FIG. 1D), wherein R₁ is defined above. These derivatives can be prepared as the last step of a solid-phase peptide synthesis before the peptide is removed from the resin by methods well known in the art. Substitutions with R₂ do not substantially decrease the ability of P₁ to bind metal ions.

In addition, P₁ and P₂ can be substituted with non-peptide functional groups that bind metal ions. These metal-binding functional groups can be attached to one or more pendent groups of the peptide, and the resulting peptide derivatives will possess one or more sites that are capable of binding metal ions, in addition to the binding site provided by P₁ and, optionally, the binding site provided by P₂. As a consequence, the ability of such peptide derivatives to bind metal ions is improved as compared to the corresponding unmodified peptide. For instance, the peptide derivative can bind two of the same type of metal ion instead of one (e.g., two Cu(II)), the peptide derivative can bind two different metal ions instead of one type of metal ion (e.g., one Cu(II) and one Fe(III)), or the peptide derivative can bind one metal ion better (e.g., with greater affinity) than the corresponding unmodified peptide.

Metal-binding functional groups include polyamines (e.g., diamines, triamines, etc.) which can bind copper. Suitable diamines include 1,2-alkyldiamines, preferably alkyl diamines wherein the alkyl contains 2-10 carbon atoms (e.g., H₂N—(CH₂)ₙ—NH₂, wherein n=2-10). Suitable diamines also include 1,2-aryldiamines, preferably benzene diamines (e.g., 1,2-diaminobenzene). Suitable diamines further include 1,2-cyclic alkane diamines. “Cyclic alkanes” are compounds containing 1-3 rings, each containing 5-7 carbon atoms. Preferably the cyclic alkane diamine is 1,2-diaminocylcohexane (cyclohexane diamine).

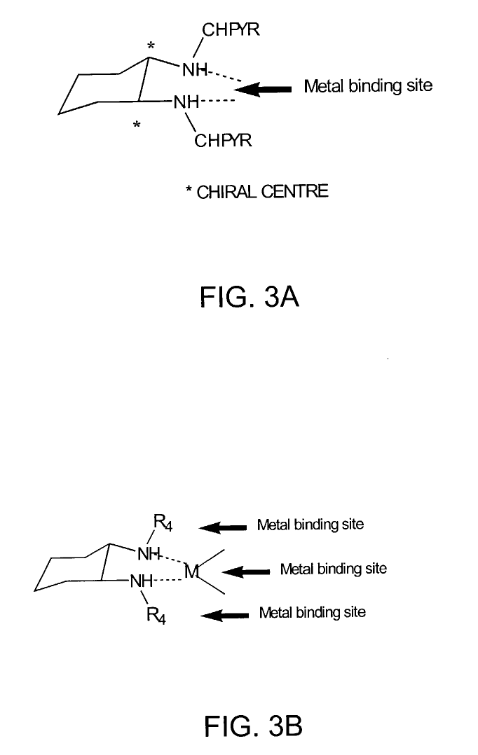

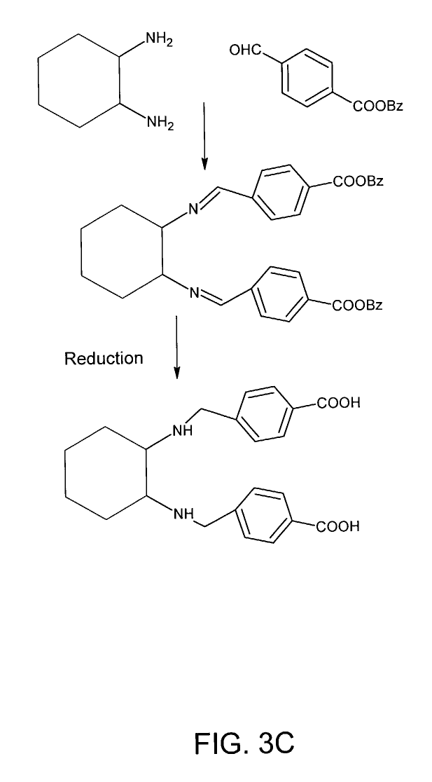

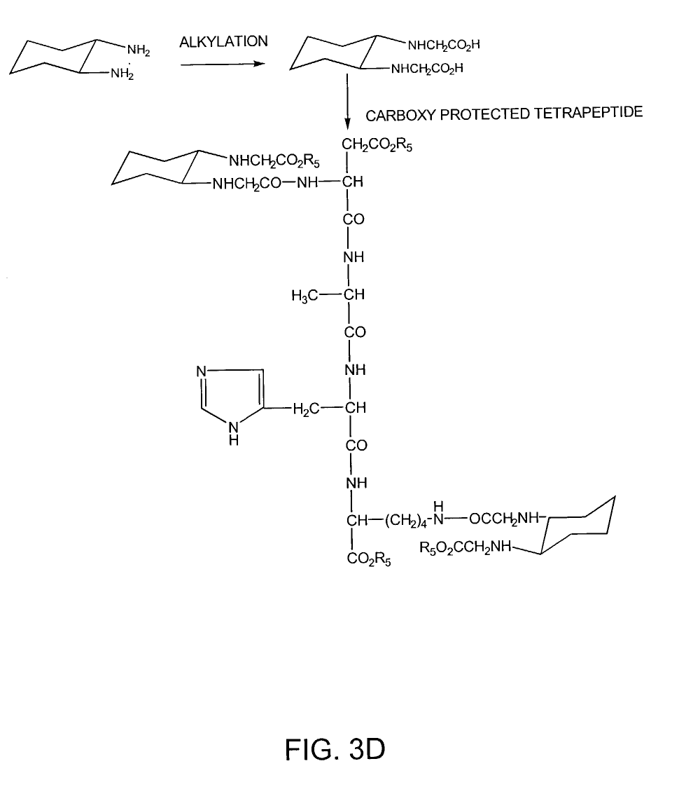

A particularly preferred diamine is 1,2-diaminocyclohexane (FIGS. 3A-B). Previous studies carried out by Rao & P. Williams (J. Chromatography A, 693, 633 (1995)) have shown that a cyclohexane diamine derivative (FIG. 3A, where PYR is pyridine) binds to a variety of metal ions. The resulting metal chelator has been successfully used to resolve amino acids and peptides, showing that the molecule has a very high affinity for a-amino acids, forming a very stable coordination complex, which is unique in many respects. 1,2-Diaminocyclohexane possesses a reactive amino functional group to which a peptide of the invention can be attached. See FIG. 3B, where M is a metal ion and at least one R₄ is -alkyl-CO-peptide, -aryl-CO-peptide, -aryl-alkyl-CO-peptide, or -alkyl-aryl-CO-peptide (see also FIGS. 3C-D). The other R₄ may be the same or may be -alkyl-COOH, -aryl-COOH, -aryl-alkyl-COOH, or alkyl-aryl-COOH. Derivatives of the type shown in FIG. 3B will have several metal-binding sites and can, therefore, be expected to bind metal ions more readily than the unsubstituted peptide. Further, due to the presence of the cyclohexane functionality, the compound will possess lipid-like characteristic which will aid its transport across lipid membranes.

Cyclohexane diamine derivatives of the peptides of the invention can be prepared by two distinct routes. The first involves initial condensation with an aldehyde followed by reduction (see FIG. 3C; in FIG. 3C Bz is benzyl). A number of aldehydes (alkyl and aryl) react readily with cyclohexane diamine at room temperature, forming an oxime. The oxime can be reduced with sodium borohydride under anaerobic conditions to give the diacid derivative. The carboxyl moieties are then reacted with the free amino groups present in carboxy-protected P₁ to give the cyclohexane diamine derivative of the peptide. The second route is a direct alkylation process which is illustrated in FIG. 3D. For example, cyclohexane diamine is treated with bromoacetic acid to give the diacetic acid derivative. The carboxyl moieties are then reacted with the free amino groups present in carboxy-protected P, to give the derivative. In FIG. 3D, R₅ is H or another peptide. When R₁ is H, the derivative can be further reacted to produce typical carboxylic acid derivatives, such as esters, by methods well known in the art. Metal binding experiments have indicated that the presence or absence of this group does not have a bearing on the metal binding capacity of the whole molecule. However, these groups would either make the molecule hydrophobic or hydrophilic, depending upon the substituent, and this may, in turn, have an effect on delivery of the molecule across membranes or to target tissues. These two synthetic routes will work for the synthesis of diamine peptide derivatives using the other diamines described above.

Additional suitable polyamines and polyamine derivatives and methods of attaching them to peptides are described in U.S. Pat. Nos. 5,101,041 and 5,650,134, the complete disclosures of which are incorporated herein by reference. Other polyamine chelators suitable for attachment to peptides are known. See, e.g., U.S. Pat. Nos. 5,422,096, 5,527,522, 5,628,982, 5,874,573, and 5,906,996 and PCT applications WO 97/44313, WO 97/49409, and WO 99/39706.

It is well known that vicinal diacids bind to metal ions, and the affinity for copper is particularly high. It is therefore envisaged that a peptide having a vicinal diacid functional group will be extremely effective in metal binding. Suitable vicinal diacids include any 1,2-alkyldiacid, such as diacetic acid (succinic acid), and any 1,2-aryldiacid.

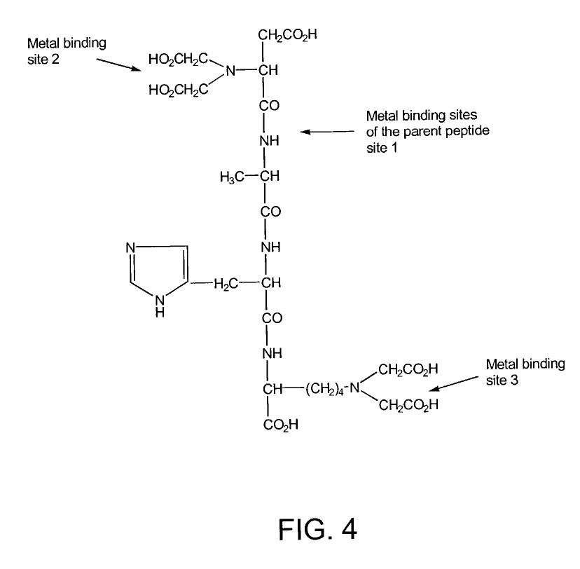

The amino groups of the peptide can be reacted with diacetic acid to produce a diacid derivative (see FIG. 4). This can be conveniently accomplished by reacting the amino groups of the resin-bound peptide with a halogenated acetic acid (e.g., bromoacetic acid or chloroacetic acid) or a halogenated acetic acid derivative (e.g., benzyloxy ester). Solid phase synthetic procedures enable removal of unreacted materials by washing with solvent. The final product is released from the resin by hydrolytic cleavage. Other diacid derivatives of the peptides of the invention can be made in the same manner.

Polyaminopolycarboxylic acids are known to bind metals, such as copper and iron. Suitable polyaminopolycarboxylic acids for making derivatives of the peptides of the invention and methods of attaching them to peptides are described in U.S. Pat. Nos. 5,807,535 and 5,650,134, and PCT application WO 93/23425, the complete disclosures of which are incorporated herein by reference. See also, U.S. Pat. No. 5,739,395.

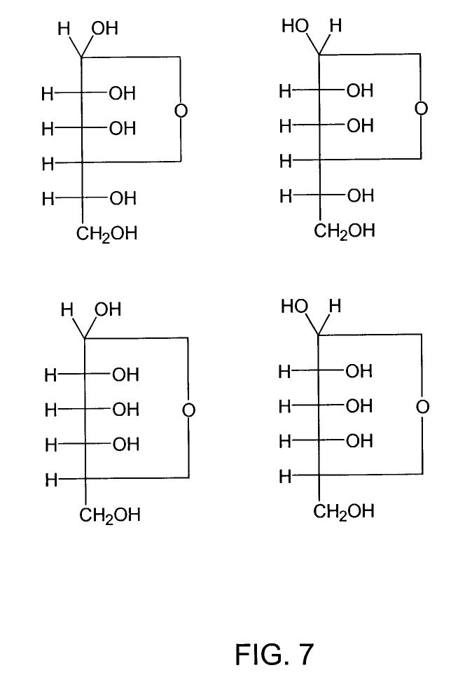

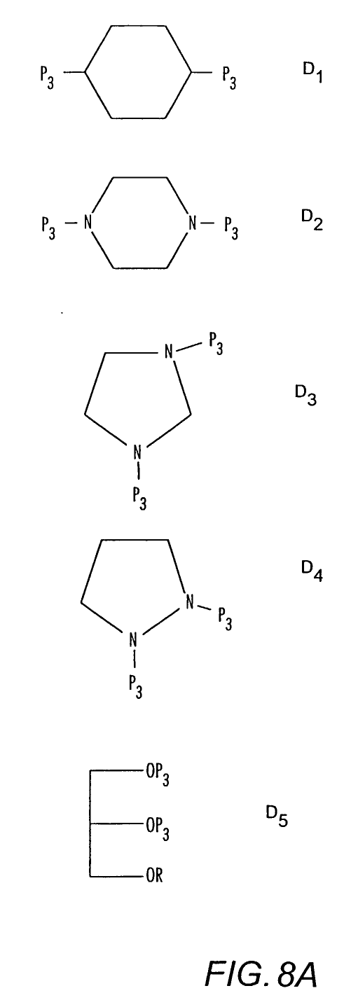

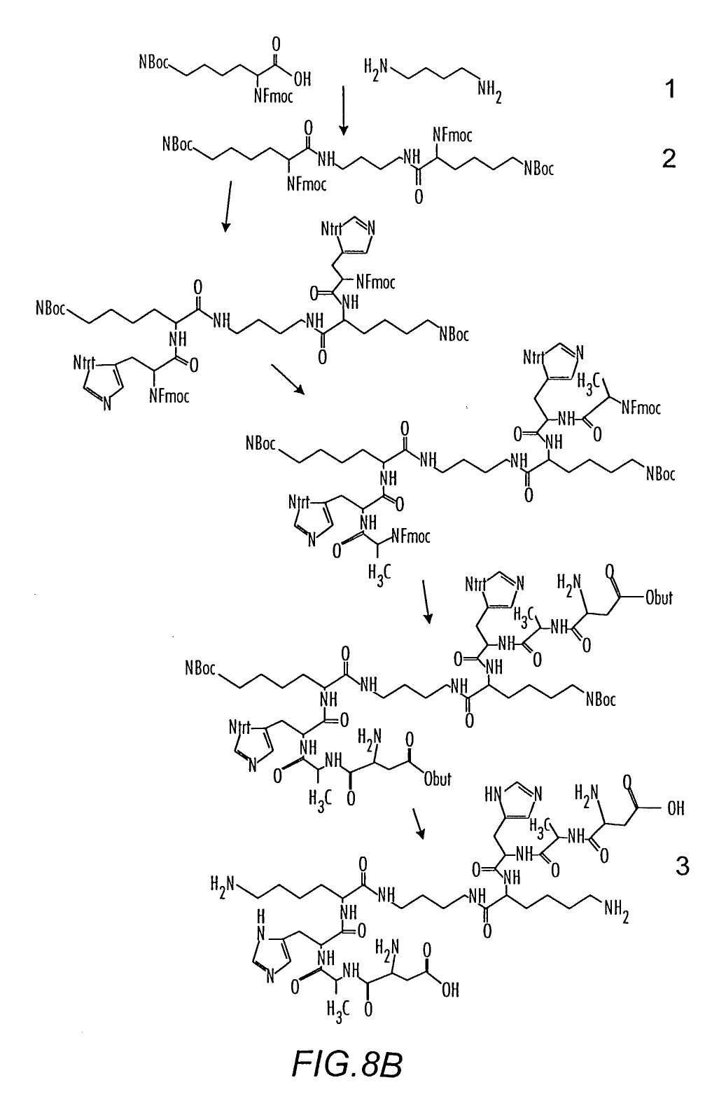

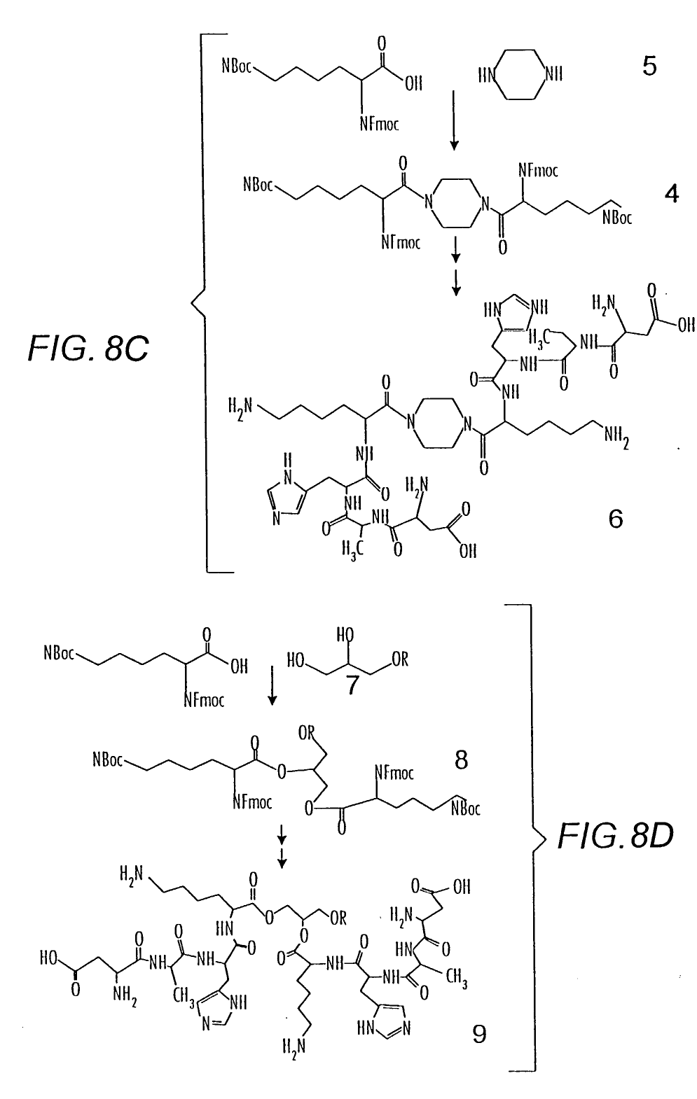

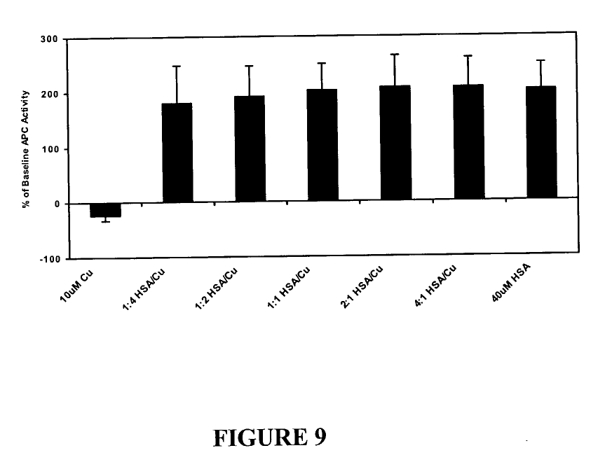

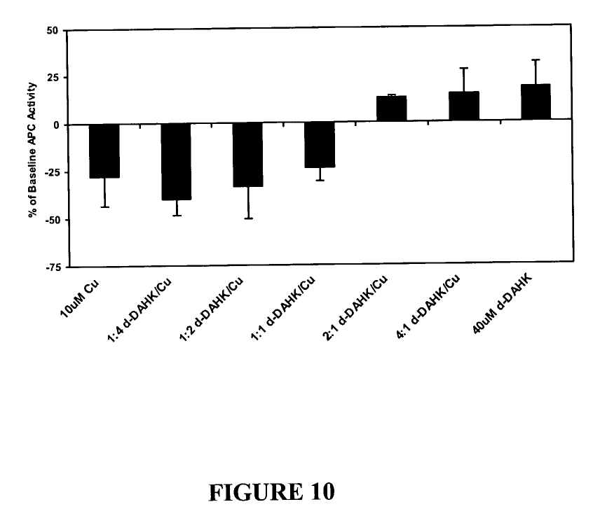

Vicinal polyhydroxyl derivatives are also included in the invention. Suitable vicinal polyhydroxyls include monosaccharides and polysaccharides (i.e., disaccharide, trisaccharide, etc.). Presently preferred are monosaccharides. See FIG. 7. The monosaccharides fall into two major categories—furanoses and pyranoses. One of the prime examples of a furanose ring system is glucose. The hydroxyl groups of glucose can be protected as benzyl or labile t-butyloxy functional groups, while leaving the aldehyde free to react with an amine group (e.g., that of lysine) of the tetrapeptide. Mild reduction/hydrolysis produces the monosaccharide peptide derivative. Other monosaccharide peptide derivatives can be prepared in this manner.

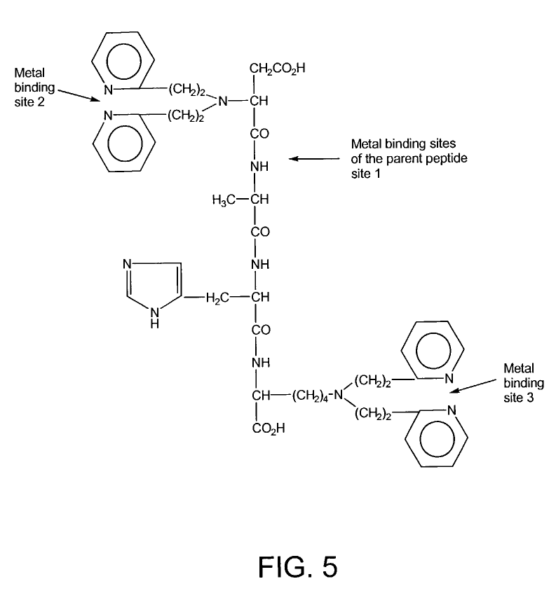

Bispyridylethylamine derivatives are known to form strong complexes with divalent metal ions. When attached to the peptide, this functional group would provide additional chelating sites for metal ions, including copper. The bispyridylethyl derivative of the tetrapeptide Asp Ala His Lys [SEQ ID NO:1] is shown in FIG. 5. It is anticipated that the metal-binding capacity of this tetrapeptide derivative will be increased by at least three-fold as compared to the underivatized peptide. The preparation of this bispyridylethylamine derivative shares some similarities with the synthesis of diacid derivatives. The two amino groups of the tetrapeptide (one at Asp and the other at Lys) are reacted with 2-bromoethylpyridine to give the tetra-substituted peptide derivative. The reaction is accomplished by reacting the resin-bound tetrapeptide with the bromoethylpyridine, followed by cleavage of the product from the resin.

Phenanthroline is another heterocyclic compound capable of binding divalent metal ions. Phenanthroline derivatives of the peptides can be synthesized in the same manner as for the bispyridylethylamine derivatives.

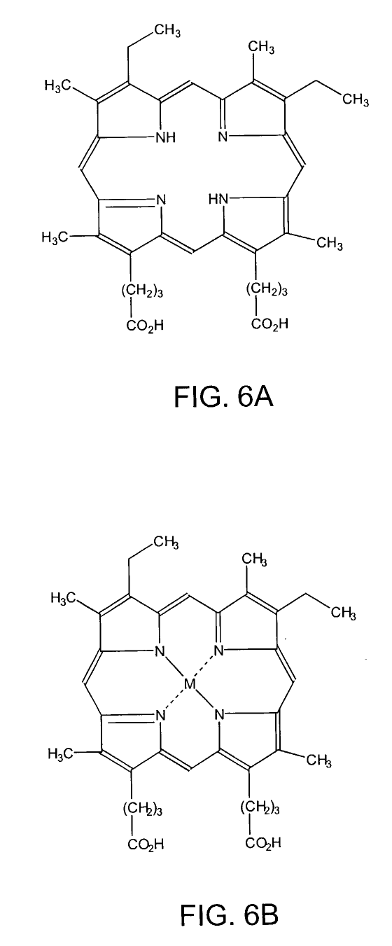

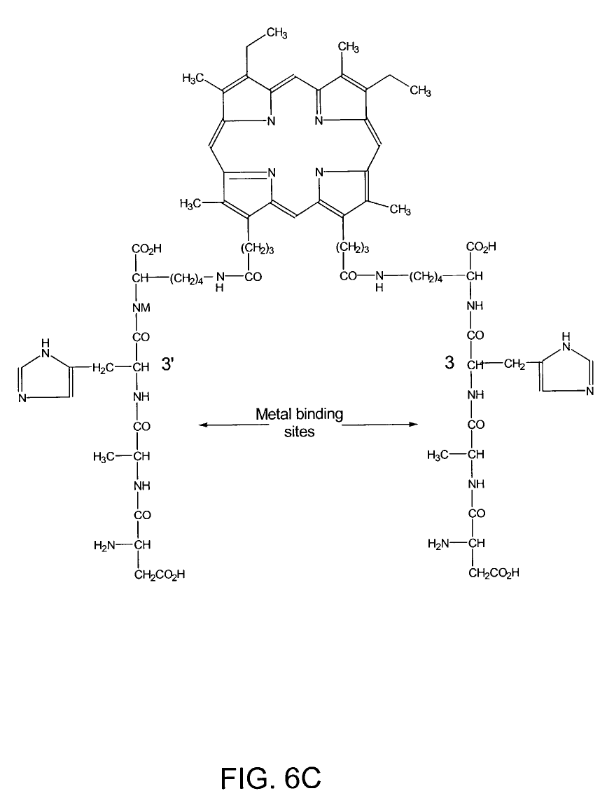

Porphyrins are a group of compounds found in all living matter and contain a tetrapyrrolic macrocycle capable of binding to metals. Heme, chlorophyll and corrins are prime examples of this class of compounds containing iron, magnesium and cobalt, respectively. Mesoporphyrin IX (FIG. 6A-B, where M is a metal ion) is derived from heme and has been observed to possess specific affinity for copper. Addition of this structure to a peptide of the invention would produce a porphyrin-peptide derivative possessing several sites for binding of copper (see FIG. 6C). In addition to their roles in metal binding, the imidazole residues at positions 3 and 3′ of the tetrapeptide shown in FIG. 6C may provide a binding site for metals other than copper, thereby stabilizing the porphyrin-metal complex. In particular, cyanocobalamine (vitamin B-12) contains cobalt as the metal in the porphyrin nucleus, and the complex is stabilized by the imidazole groups. On the basis of this analogy it is anticipated that the porphyrin-tetrapeptide derivative would bind cobalt (or other metals) at normal physiological conditions in the prophyrin nucleus and that the complex would be stabilized by the His imidazole groups.

To prepare the porphyrin-peptide derivative shown in FIG. 6C, the carboxyl groups of mesoporphyrin IX can be activated and coupled with the amino groups of the peptide employing standard solid-phase peptide synthesis. Typically, the free amino group of the lysine residue of the resin-bound peptide can be coupled with carboxy activated porphyrin nucleus. The condensation product can be cleaved off the resin using standard methods. This method can be used to synthesize other porphyrin derivatives of peptides of the invention.

Other suitable porphyrins and macrocyclic chelators and methods of attaching them to peptides are described in U.S. Pat. Nos. 5,994,339 and 5,087,696, the complete disclosures of which are incorporated herein by reference. Other porphyrins and macrocyclic chelators that could be attached to peptides are known. See, e.g., U.S. Pat. Nos. 5,422,096, 5,527,522, 5,628,982, 5,637,311, 5,874,573, and 6,004,953, PCT applications WO 97/44313 and WO 99/39706.

A variety of additional metal chelators and methods of attaching them to proteins are described in U.S. Pat. No. 5,683,907, the complete disclosure of which is incorporated herein by reference.

Dithiocarbamates are known to bind metals, including iron. Suitable dithiocarbamates for making derivatives of the peptides of the invention are described in U.S. Pat. Nos. 5,380,747 and 5,922,761, the complete disclosures of which are incorporated herein by reference.

Hydroxypyridones are also known to be iron chelators. Suitable hydroxypyridones for making derivatives of the peptides of the invention are described in U.S. Pat. Nos. 4,912,118 and 5,104,865 and PCT application WO 98/54138, the complete disclosures of which are incorporated herein by reference.

Additional non-peptide metal chelators are known in the art or will be developed. Methods of attaching chemical compounds to proteins and peptides are well known in the art, and attaching non-peptide metal chelators to the peptides of the invention is within the skill in the art. See, e.g., those patents cited above describing such attachment methods.

As can be appreciated, the non-peptide metal-binding functional groups could be attached to another peptide in the same manner as they are to peptide P₁-P₂. The resulting peptide derivatives would contain one or more non-peptide metal-binding functional groups attached to a peptide which could, optionally, comprise a metal-binding site of a peptide (as described above, the sequences of many metal-binding peptides, including copper-binding peptides, are known). At least one of the metal-binding functional group(s) or the optional metal-binding site of the peptide must bind copper. Preferably, the peptide contains from 2-10, more preferably 3-5, amino acids. Preferably the peptide contains one or more D-amino acids; most preferably all of the amino acids of the peptide are D-amino acids. These peptides and derivatives of them having one or more non-peptide metal-binding functional groups attached to them can be prepared in the same ways as described above for peptides P₁-P₂ and similar derivatives of them.

Another preferred group of copper chelators for use in the practice of the method of the invention are peptide dimers of the formula:

P₃-L-P₃.

P₃ is any peptide capable of binding copper, and each P₃ may be the same or different. Each P₃ preferably contains 2-10, more preferably 3-5, amino acids. As described above, copper-binding peptides are known, and each P₃ may comprise the sequence of one or more of the copper-binding sites of these peptides. Although each P₃ may be substituted as described above for P₁ and P₂, including with a non-peptide, metal-binding functional group, both P₃ peptides are preferably unsubstituted. P₃ may also comprise any amino acid sequence substituted with a non-peptide, copper-binding functional group as described above to provide the copper-binding capability of P₃. Preferably, each P₃ is an unsubstituted copper-binding peptide (i.e., an unsubstituted peptide comprising a peptide sequence which binds copper). Most preferably, one or both of the P₃ groups is P₁ (i.e., the dimers have the sequence P₃-L-P₁, P₁-L-P₃ or, most preferably, P₁-L-P₁). P₁ is defined above.

L is a linker which is attached to the C-terminal amino acid of each P₃. L may be any physiologically-acceptable chemical group which can connect the two P₃ peptides through their C-terminal amino acids. By “physiologically-acceptable” is meant that a peptide dimer containing the linker L is not toxic to an animal (including a human) or an organ to which the peptide dimer is administered as a result of the inclusion of the linker L in the peptide dimer. Preferably, L links the two P₃ groups so that they can cooperatively bind metal ions (similar to a 2:1 peptide:metal complex). L is also preferably neutral. Most preferably, L is a straight-chain or branched-chain alkane or alkene residue containing from 1-18, preferably from 2-8, carbon atoms (e.g., —CH₂—, —CH₂CH₂—, —CH₂CH₂CH₂—, —CH₂CH₂(CH₃)CH₂—, —CHCH—, etc.) or a cyclic alkane or alkene residue containing from 3-8, preferably from 5-6, carbon atoms (see FIG. 19A, compound D₁), preferably attached to a P₃ by means of an amide linkage. Such linkers are particularly preferred because they impart hydrophobicity to the peptide dimers. In another preferred embodiment, L is a nitrogen-containing heterocyclic alkane residue (see FIG. 19A, compounds D₂, D₃ and D₄), preferably a piperazide (see FIG. 19A, compound D₂). In another preferred embodiment L is a glyceryl ester (see FIG. 19A, compound D₅; in formula D₅, R is an alkyl or aryl preferably containing 1-6 carbon atoms). Finally, L could be a metal-binding porphyrin (see FIG. 6C). These preferred linkers L will allow the two peptides P₃ to bind metal ions cooperatively and are biocompatible, and the peptide dimers containing these preferred linkers can be made easily and in large quantities. By “biocompatible” is meant that a peptide dimer containing the linker L does not produce any undesirable side-effects due to the linker L in an animal (including a human) to which the peptide dimer is administered.

Methods of synthesizing the peptide dimers are illustrated in FIGS. 19B-D. In general, the C-terminal amino acids (protected by methods and protecting groups well known in the art) of the two P₃ groups are attached to L, and the resulting amino acid dimers used in standard peptide synthetic methods to make the peptide dimers.

For instance, a peptide dimer, where each peptide has the sequence Asp Ala His Lys, [SEQ ID NO:1] can be synthesized by coupling protected lysines to a free diamine functional group, either as an acid chloride or by using standard coupling agents used in peptide synthesis (see FIGS. 19B-C). Many suitable diamines are available commercially or suitable diamines can be readily synthesized by methods known in the art.

For instance, the lysine dimer 2 (FIG. 19B) can be prepared as follows. To a stirred solution of 9-fluorenylmethyloxycarbonyl (Fmoc)- and t-benzyloxycarbonyl(Boc)-protected D-Lys (Fmoc-D-Lys(Boc)-OH) (20 mmole) in dry dimethylformamide (DMF; 100 mL; dry argon flushed) are added butane-1,4-diamine 1 and 2-(1H-benzotriazole-1-yl)-1,2,3,3-tetramethyluroniumtetrafluoroborate (TBTU; 0.5 mmole). The solution is stirred for 36 hours at room temperature. The bis-protected lysine 2 is isolated by flash chromatography over silica and elution with mixtures of ethyl acetate/methanol. The peptide dimer 3 is then prepared from the protected lysine dimer 2 employing classical peptide synthesis methodology (see FIG. 19B).

Another peptide dimer, where each peptide has the sequence Asp Ala His Lys [SEQ ID NO:1], can be synthesized as follows. First, a different protected lysine dimer 4 is synthesized by acylating the two amino centers of a piperazine 5 (see FIG. 19C; see also Chambrier et al., Proc. Natl. Acad. Sci., 96, 10824-10829 (1999)). Then, the remainder of the amino acid residues are added employing standard peptide synthesis methodology to give the peptide dimer 6 (see FIG. 19C).

Peptide dimers, where each peptide has the sequence Asp Ala His Lys [SEQ ID NO:1] and where L is a glyceryl ester, can be synthesized as follows. The 3-substituted propane-1,2-diols of formula 7 in FIG. 19D, wherein R is an alkyl or aryl, are commercially available. A lysine diester 8, wherein R is methyl, can be prepared as follows (see FIG. 19D). To a stirred solution of Fmoc-D-Lys(Boc)-OH (20 mmole) in dry toluene (100 mL; dry argon flushed) is added 3-methoxypropane-1,2-diol (200 mmole) and imidazole (15 mmole). The solution is stirred for 36 hours at room temperature. The solvent is removed in vacuo, and the residue is dissolved in ethyl acetate. This solution is washed with citric acid solution (2%), water, 0.5 N NaHCO₃ solution, and again with water; then the organic layer is dried over magnesium sulphate (removal of the solvent gives a pale yellow residue). The bis-protected lysine 8 is isolated by flash chromatography over silica and elution with mixtures of ethyl acetate/methanol. The peptide dimer 9 is then prepared from the protected lysine dimer 8 employing classical peptide synthesis methodology (see FIG. 19D).

The physiologically-acceptable salts of the metal-binding compounds are also included in the invention. Physiologically-acceptable salts include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, benzoic, salicylic, and the like) or bases (such as the hydroxide, carbonate or bicarbonate of a pharmaceutically-acceptable metal cation). The salts are prepared in a conventional manner, e.g., by neutralizing the free base form of the compound with an acid.

In one embodiment of the invention, an effective amount of a copper chelator is administered to an animal in need of treatment with APC. Preferably, the animal is a mammal, such as a rabbit, goat, dog, cat, horse or human, most preferably a human. In addition to the chelator, an effective amount of APC, protein C, agent that increases the synthesis of protein C, and/or protein C activator is administered to the animal (see above). The copper chelator can be administered prior to, at the same time and/or after the APC, protein C, agent that increases the synthesis of protein C, and/or protein C activator is administered. Preferably, the copper chelator is administered prior to administration of APC, protein C, agent that increases the synthesis of protein C, and/or protein C activator, and administration of the chelator is continued during the administration of the APC, protein C, agent that increases the synthesis of protein C, and/or protein C activator. If the copper chelator is administered at the same time as the APC, protein C, agent that increases the synthesis of protein C, and/or protein C activator, all of the compounds can be administered in admixture with each other or separately.

Effective dosage forms, modes of administration and dosage amounts for the various copper chelators of the invention may be determined empirically, and making such determinations is within the skill of the art. It has been found that an effective dosage is from about 2 to about 200 mg/kg, preferably from about 10 to about 40 mg/kg, most preferably about 20 mg/kg. However, it is understood by those skilled in the art that the dosage amount will vary with the particular chelator employed, the disease or condition to be treated, the severity of the disease or condition, the route(s) of administration, the rate of excretion of the compound, the duration of the treatment, the identify of any other drugs being administered to the animal, the age, size and species of the animal, and like factors known in the medical and veterinary arts. In general, a suitable daily dose of a chelator of the present invention will be that amount of the chelator which is the lowest dose effective to produce a therapeutic effect. However, the daily dosage will be determined by an attending physician or veterinarian within the scope of sound medical judgment. If desired, the effective daily dose may be administered as two, three, four, five, six or more sub-doses, administered separately at appropriate intervals throughout the day, or may be administered as a continuous infusion. Administration of the chelator should be continued until an acceptable response is achieved.

The chelators of the present invention may be administered to an animal patient for therapy by any suitable route of administration, including orally, nasally, rectally, vaginally, parenterally (e.g., intravenously, intraspinally, intraperitoneally, subcutaneously, or intramuscularly), intracistemally, transdermally, transmucosally, intracranially, intracerebrally, and topically (including buccally and sublingually). The preferred route of administration is parenterally.

While it is possible for a chelator of the present invention to be administered alone, it is preferable to administer the compound as a pharmaceutical formulation (composition). The pharmaceutical compositions of the invention comprise a chelator or chelators of the invention as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the animal. Pharmaceutically-acceptable carriers are well known in the art. Regardless of the route of administration selected, the compounds of the present invention are formulated into pharmaceutically-acceptable dosage forms by conventional methods known to those of skill in the art. See, e.g., Remington's Pharmaceutical Sciences.

Pharmaceutical compositions of this invention suitable for parenteral administrations comprise one or more chelators of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a chelator or chelators of the present invention as an active ingredient. A chelator or chelators of the present invention may also be administered as bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

Liquid dosage forms for oral administration of the chelators of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compound(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical, transdermal or transmucosal administration of a chelator of this invention include powders, sprays, ointments, pastes, creams, lotions, gels, solutions, patches, drops and inhalants. The chelator or chelators may be mixed under sterile conditions with a pharmaceutically-acceptable carrier, and with any buffers, or propellants which may be required.

The ointments, pastes, creams and gels may contain, in addition to a chelator or chelators of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a chelator or chelators of this invention, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

The active ingredient (i.e., a chelator or chelators of the invention) may also be delivered through the skin using conventional transdermal drug delivery systems, i.e., transdermal patches, wherein the active ingredient is typically contained within a laminated structure that serves as a drug delivery device to be affixed to the skin. In such a structure, the active ingredient is typically contained in a layer, or “reservoir,” underlying an upper backing layer. The laminated device may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active ingredient and any other materials that are present. The backing layer may be either occlusive or nonocclusive, depending on whether it is desired that the skin become hydrated during drug delivery. The backing is preferably made of a sheet or film of a preferably flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery devices may be fabricated using conventional techniques, known in the art, for example by casting a fluid admixture of adhesive, active ingredient and vehicle onto the backing layer, followed by lamination of the release liner. Similarly, the adhesive mixture may be cast onto the release liner, followed by lamination of the backing layer. Alternatively, the drug reservoir may be prepared in the absence of active ingredient or excipient, and then loaded by “soaking” in a drug/vehicle mixture.

The laminated transdermal drug delivery systems may, in addition, contain a skin permeation enhancer. That is, because the inherent permeability of the skin to some active ingredients may be too low to allow therapeutic levels of the drug to pass through a reasonably sized area of unbroken skin, it is necessary to coadminister a skin permeation enhancer with such drugs. Suitable enhancers are well known in the art.

The pharmaceutical compositions of the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art.

In another embodiment of the invention, a composition comprising the APC, protein C, agent that increases the synthesis of protein C, and/or protein C activator is contacted with the copper chelator prior to administration of an effective amount of the APC, protein C, agent that increases the synthesis of protein C, and/or protein C activator in order to chelate any copper present in the composition. Suitable contacting conditions can be determined empirically and doing so is within the skill in the art. For instance, contacting can be accomplished by simply combining a solution of the chelator and a solution of the other compound(s) and incubating them employing conditions (e.g., time, temperature, and pH) sufficient to allow the chelator to bind any copper in the compositions. The copper chelator can be removed prior to administration (e.g., use of a copper chelator in the manufacture of a copper-free composition) or, preferably, is administered along with the APC, protein C, agent that increases the synthesis of the protein C, and/or protein C activator. The copper chelator can be removed in a variety of ways, including using an affinity column comprising an antibody specific for the chelator.

It is to be noted that “a” or “an” entity refers to one or more of that entity. For example, “a cell” refers to one or more cells.

### EXAMPLES

### Example 1

This example describes an in vitro study which investigated whether copper inhibits activated protein C (APC) anticoagulant activity. As shown below, after a thirty-minute incubation period, copper inhibited APC anticoagulant activity.

Human albumin has one high-affinity binding site for copper at its N-terminus and numerous non-specific copper binding sites elsewhere. Linder et al., Biochemistry of Copper (Plenum Press, New York, 1991); Peters, All About Albumin: Biochemistry, Genetics, and Medical Applications (Academic Press, San Diego, 1996); Sadler et al., Eur. J. Biochem. 220, 193-200 (1994); Bar-Or et al., Eur. J. Biochem. 268, 42-47 (2001). Albumin sequesters free copper ions, and the N-terminal copper-binding site has been shown to prevent copper-induced formation of reactive oxygen species. Bar-Or et al., Biochem. Biophys. Res. Commun. 284, 856-862 (2001); Bar-Or et al., Biochem. Biophys. Res. Commun. 282, 356-360 (2001).

The in vitro study described in this example also investigated whether the copper-induced APC inhibition could be prevented or reversed by human serum albumin (HSA) or an analogue of the human albumin N-terminus copper-binding site, D-Asp D-Ala D-His D-Lys (d-DAHK). As shown below, after a thirty-minute incubation period, both HSA and d-DAHK demonstrated a protective effect against copper-induced inhibition of APC activity, suggesting that copper chelation would enhance APC therapeutic efficacy.

Materials. The human albumin N-terminus analogue, d-DAHK, was synthesized by Bowman Research, Ltd (Newport, Wales, U.K.). The assay for APC anticoagulant activity was the Accucolor™ calorimetric assay (Sigma Diagnostics, St. Louis, Mo.). APC (Sigma, product number P₂₂₀₀), HSA (Sigma, product number A1653), and all other chemicals were obtained from Sigma (St. Louis, Mo.). Copper content of APC was determined by graphite furnace atomic absorption (Galbraith Laboratories, Knoxville, Tenn.).

APC activity assay. The APC assay, originally designed for plasma samples (Francis et al., Am. J. Clin. Pathol. 87, 619-625 (1987)) was modified for use in a clean, aqueous environment. Solutions of CuCl₂, HSA, and d-DAHK were prepared in 20 mM KH₂PO₄ buffer (pH 7.4). Experiments were performed in a 96-well plate. APC (2 mg/L) was added to the following solutions: a) 20 mM KH₂PO₄ buffer alone, b) 10 μM CuCl₂, c) 40 μM HSA, d) 40 μM d-DAHK, e) HSA:CuCl₂ together in ratios of 1:4, 1:2, 1:1, 2:1, and 4:1 and f) d-DAHK:CuCl₂ together in ratios of 1:4, 1:2, 1:1, 2:1, and 4:1 (n=3, in duplicate, for each) and incubated for 30 minutes at 37° C. Protein C substrate (2 mg/L) was then added to each well and incubated for ten minutes at 37° C. Concentrated acetic acid was added to each well to stop the reaction and the results were read at 410 nm on a microplate fluorescence reader (Model FL600, Bio-Tek Instruments, Inc., Winooski, Vt.). APC activity for copper alone, HSA alone, d-DAHK alone, and all combinations of HSA:CuCl₂, and d-DAHK:CuCl₂ were expressed as the mean percent change (±standard deviation) from baseline APC activity using buffer alone.

Copper Inhibition of APC Incubating CuCl₂ with APC in two separate experiments demonstrated a decrease of 27.9%±15.5% (FIG. 1) and 24.1%±9.7% (FIG. 2) in APC activity compared to baseline (overall mean decrease 26.0%±11.8%). Copper has previously been used to bind APC during metal-affinity chromatography techniques. Wu et al., Biotechnol. Prog. 15, 928-931 (1999). However, as far as is know, the present study is the first to provide evidence that copper inhibits in vitro APC anticoagulant activity.

Copper is an essential trace metal that is tightly regulated by plasma proteins under normal conditions. Acidic conditions in vitro are known to cause free copper ions to be released from ceruloplasmin and other proteins, and free copper is released in vivo during conditions involving ischemia and acidosis. Linder et al., Biochemistry of Copper (Plenum Press, New York, 1991); Berenshtein et al., J. Mol. Cell. Cardiol. 29, 3025-3034 (1997); Lamb et al., FEBS Lett. 338, 122-126 (1994). Ischemia and acidosis frequently accompany septic shock and often occur early in sepsis (without signs of shock) due to increased tissue oxygen requirements, impaired oxygen extraction, and maldistribution of blood flow. Pastores et al., Am. J. Gastroenterol. 91, 1697-1710 (1996). Thus, free copper is readily available during sepsis to inhibit the activity of both endogenous and therapeutically administered APC, implying that copper sequestration may enhance the therapeutic efficacy of APC or of endogenously inactivated APC.

HSA Prevents Copper Inhibition of APC. Incubating HSA with APC resulted in a 202.1%±48.1% increase over baseline APC activity (FIG. 1). Such a dramatic increase in APC activity suggested that the APC used in the experiments may have contained copper. Analysis of the copper content of the APC stock solution (300 mg/L; 5.45 μM) used in all experiments in the present study was determined to contain 88.5 μg/L (1.39 μM) copper. Numerous available copper-binding sites of HSA may have removed the APC-bound copper to increase APC activity over baseline). Linder et al., Biochemistry of Copper (Plenum Press, New York, 1991); Peters, All About Albumin: Biochemistry, Genetics, and Medical Applications (Academic Press, San Diego, 1996). Other potential HSA-APC interactions, such as conformational changes exposing active sites on APC or a substrate-like activity of HSA, could also have increased APC activity. Various ratios of HSA:CuCl₂ consistently prevented any copper-induced inhibition of APC and resulted in dramatically increased APC activity ranging from 180.0%±68.2% to 207.1%±53.3% over baseline (FIG. 1). Hypoalbuminemia is often reported in sepsis and may be due to increased albumin catabolism, extravascular escape, and to a lesser extent by decreased albumin synthesis. Ruot et al., Am. J. Physiol. Endocrinol. Metab. 279, E244-251 (2000). The administration of human albumin for septic shock and intestinal ischemic shock has been reported to improve hemodynamic parameters and survival when compared to electrolyte solution alone. Dawidson et al., Crit. Care Med. 18, 60-66 (1990); Rackow et al., Crit. Care Med. 11, 839-850 (1983); Ottosson et al., Crit. Care Med. 17, 772-779 (1989). Deferoxamine, which chelates both copper and iron, was also reported to be beneficial in animal models of sepsis. Moch et al., Shock 4, 425-432 (1995); Jung et al., J. Hepatol. 33, 387-394 (2000). Theoretically, in view of the results presented here, part of the benefit of administering albumin or a metal chelator for sepsis and shock might be explained by the prevention of copper-induced APC inhibition or reactivation of endogenously inactivated APC.

d-DAHK Prevents Copper Inhibition ofAPC. Incubating d-DAHK with APC resulted in an 18.2%±13.0% increase over baseline APC activity (FIG. 2). Ratios of 2:1 and 4:1 d-DAHK:CuCl₂ increased APC activity over baseline (12.9%±1.1%, and 14.8%±12.7%, respectively), while lower d-DAHK:CuCl₂ ratios demonstrated no significant protection of copper-induced inhibition of APC (FIG. 2). That observation is consistent with our previous reports that d-DAHK effectively binds free copper in a ratio of at least 2:1 d-DAHK:CuCl₂. Bar-Or et al., Eur. J. Biochem. 268, 42-47 (2001); Bar-Or et al., Biochem. Biophys. Res. Commun. 284, 856-862 (2001); Bar-Or et al., Biochem. Biophys. Res. Commun. 282, 356-360 (2001). The maximal effect of d-DAHK on APC alone (FIG. 2) resulted in an 18.2% increase in activity above baseline, which corresponds to the independently measured amount of copper in APC (1.39 μM copper for 5.45 μM APC, 1:4) being chelated by d-DAHK.

Despite advances in critical care, severe sepsis is a relatively common and frequently fatal disease that is more likely to be fatal in elderly patients. Angus et al., Crit. Care Med. 29, 1303-1310(2001). In a Phase 3 clinical trial for the treatment of severe sepsis, recombinant human APC reduced 28-day mortality rates from 31% to 25%; however, a substantial number of patients receiving APC did not have any beneficial effect. Bernard et al., N. Engl. J. Med. 344, 699-709 (2001). Intravenous human recombinant APC is cleared from the plasma of healthy subjects by proteolytic enzymes in less than 15 minutes and, according to the manufacturer, up to 50% faster in patients with severe sepsis. Bernard et al., Crit. Care Med. 29, 2051-2059 (2001); Gruber et al., Circulation 82, 578-585 (1990); Okajima et al., Thromb. Haemost. 63, 48-53 (1990); Yan et al., Crit. Care Med 29, S69-74 (2001); Grinnell et al., Crit. Care Med. 29, S53-60; discussion S60-1 (2001). Thus, current clinical guidelines recommend that intravenous APC be administrated continuously over four days. Bernard et al., N. Engl. J. Med. 344, 699-709 (2001). Preventing copper-induced inhibition of APC by sequestering free copper with albumin or d-DAHK might allow lower doses of APC to be administered over a shorter period of time, while maintaining, or even enhancing, the clinical benefit of APC.

In conclusion, these results suggest that copper partially inhibits APC anticoagulant activity in vitro and that HSA and d-DAHK, an analogue of the high affinity copper-binding site of human albumin, prevent copper-induced inhibition of APC. Free copper that is mobilized during the ischemia and acidosis accompanying sepsis may contribute to the deactivation of APC, reducing its clinical benefit.

We claim:

1. A method of treating an animal in need of treatment with activated protein C (APC), the method comprising administering to the animal:
an effective amount of a copper chelator; and
an effective amount of one of the following:
(a) APC;
(b) protein C, an agent that increases the synthesis of protein C in the animal, or both;
(c) an activator of protein C; or
(d) a combination of one or more of (a), (b) and (c).

2. The method of claim 1 wherein the chelator is human albumin or a fragment thereof comprising the N-terminal copper-binding sequence Asp Ala His.

3. The method of claim 1 wherein the chelator is a peptide having the formula:
P₁-P₂,
wherein:
P₁ is:
Xaa₁ Xaa₂His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, omithine, phenylalanine, tyrosine, tryptophan, cysteine, methionine, or a-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

4. The method of claim 3 wherein Xaa₁ is aspartic acid, glutamic acid, arginine, threonine, or α-hydroxymethylserine.

5. The method of claim 3 wherein Xaa₂ is glycine, alanine, valine, leucine, isoleucine, threonine, serine, asparagine, methionine, histidine or a-hydroxymethylserine.

6. The method of claim 3 wherein Xaa₃ is lysine.

7. The method of claim 3 wherein Xaa₁ is aspartic acid, glutamic acid, arginine, threonine, or a-hydroxymethylserine, Xaa₂ is glycine, alanine, valine, leucine, isoleucine, threonine, serine, asparagine, methionine, histidine or a-hydroxymethylserine, and Xaa₃ is lysine.

8. The method of claim 7 wherein Xaa₁ is aspartic acid or glutamic acid and Xaa₂ is alanine, glycine, valine, threonine, serine, leucine, or a-hydroxymethylserine.

9. The method of claim 8 wherein Xaa₂ is alanine, threonine, leucine, or α-hydroxymethylserine.

10. The method of claim 9 wherein Xaa₁ is aspartic acid and Xaa₂ is alanine.

11. The method of claim 3 wherein n is 0-10.

12. The method of claim 11 wherein n is 0-5.

13. The method of claim 12 wherein n is 0.

14 The method of claim 3 wherein P₂ comprises a metal-binding sequence.

15. The method of claim 14 wherein P₂ comprises one of the following sequences:
(Xaa₄)ₘ Xaa₃ His Xaa₂ Xaa₅,
(Xaa₄)ₘ His Xaa₂ Xaa₅,
(Xaa₄)ₘ Xaa₅ Xaa₂ His Xaa₃, or
(Xaa₄)ₘ Xaa₅ Xaa₂ His,
wherein Xaa₅ is an amino acid having a free side-chain —NH₂ and m is 0-5.

16. The method of claim 15 wherein Xaa₅ is Orn or Lys.

17. The method of claim 14 wherein P₂ comprises one of the following sequences:
[(Xaa₄)ₘXaa₅Xaa₂HisXaa₃]ᵣ,
[(Xaa₄)ₘXaa₅Xaa₂His]ᵣ,
[(Xaa₄)ₘXaa₅Xaa₂HisXaa₃(Xaa₄)ₘXaa₅Xaa₂His]ᵣ, or
[(Xaa₄)ₘXaa₅Xaa₂His(Xaa₄)ₘXaa₅Xaa₂HisXaa₃]ᵣ,
wherein Xaa₅ is an amino acid having a free side-chain —NH₂, m is 0-5 and r is 2-100.

18. The method of claim 14 wherein P₂ comprises a sequence which binds Cu(I).

19. The method of claim 18 wherein P₂ comprises one of the following sequences:
Met Xaa₄ Met,
Met Xaa₄ Xaa₄ Met,
Cys Cys,
Cys Xaa₄ Cys,
Cys Xaa₄ Xaa₄ Cys,
Met Xaa₄ Cys Xaa₄ Xaa₄ Cys,
Gly Met Xaa₄ Cys Xaa₄ Xaa₄ Cys [SEQ ID NO:3],
Gly Met Thr Cys Xaa₄ Xaa₄ Cys [SEQ ID NO:4],
Gly Met Thr Cys Ala Asn Cys [SEQ ID NO:5], or
γ-Glu Cys Gly.

20. The method of claim 19 wherein P₂ is Gly Met Thr Cys Ala Asn Cys [SEQ ID NO: 5].

21. The method of claim 3 wherein P₂ comprises a sequence which enhances the ability of the peptide to penetrate cell membranes, reach target tissues, or both.

22. The method of claim 21 wherein P₂ is hydrophobic or an arginine oligomer.

23. The method of claim 3 wherein at least one of the amino acids of P₁ other than β-alanine, when present, is a D-amino acid.

24. The method of claim 23 wherein Xaa₁ is a D-amino acid, His is a D-amino acid, or both Xaa₁ and His are D-amino acids.

25. The method of claim 24 wherein all of the amino acids of P₁ other than β-alanine, when present, are D-amino acids.

26. The method of claim 23 wherein at least 50% of the amino acids of P₂ are D-amino acids.

27. The method of claim 24 wherein at least 50% of the amino acids of P₂ are D-amino acids.

28. The method of claim 25 wherein at least 50% of the amino acids of P₂ are D-amino acids.

29. The method of claim 3 wherein at least one amino acid of P₁, at least one amino acid of P₂, or at least one amino acid of P₁ and at least one amino acid of P₂, is substituted with (a) a substituent that increases the lipophilicity of the peptide without altering the ability of P₁ to bind copper ions, (b) a substituent that protects the peptide from proteolytic enzymes without altering the ability of P₁ to bind copper ions, or (c) a substituent which is a non-peptide, metal-binding functional group that does not alter the ability of P₁ to bind copper ions.

30. The method of claim 29 wherein n is 0 and P₁ has one of the following formulas: 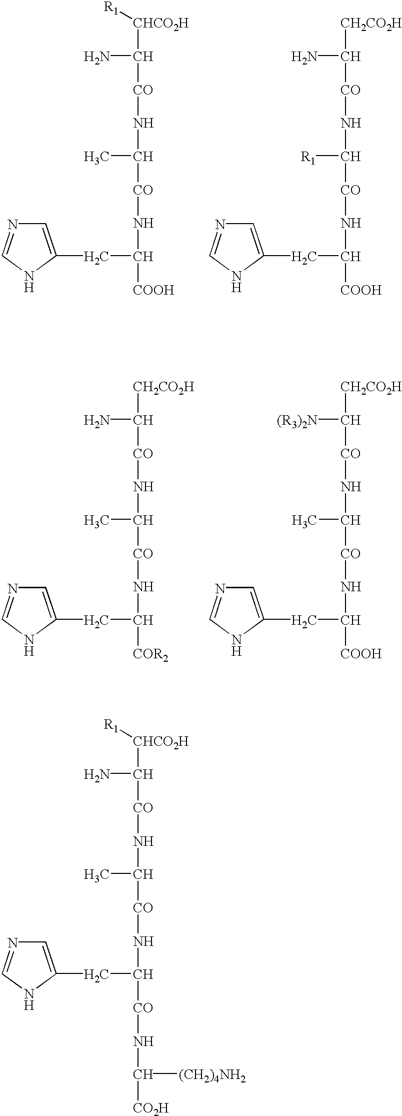
wherein:
R₁ is an alkyl, aryl, or heteroaryl;
R₂ is —NH₂, —NHR₁, N(R₁)₂, —OR₁, or R₁; and
R₃ is H, a non-peptide, metal-binding functional group or the two R₃ groups together form a non-peptide, metal-binding functional group.

31. The method of claim 1 wherein the chelator is a peptide dimer having the formula:
P₃-L-P₃,
wherein:
each P₃ may be the same or different and is a peptide which is capable of binding copper; and
L is a chemical group which connects the two P₃ peptides through their C-terminal amino acids.

32. The method of claim 31 wherein each P₃ contains 2-10 amino acids.

33. The method of claim 31 wherein at least one P₃ is P₁, wherein P₁ is:
Xaa₁ Xaa₂His: or
Xaa₁ Xaa₂ His Xaa₃; and
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine; and
Xaa₃ is glycine, alanine, valine, lysine, arginine, ornithine, aspartic acid, glutamic acid, asparagine, glutamine or tryptophan.

34. The method of claim 33 wherein Xaal is aspartic acid, glutamic acid, arginine, threonine, or a-hydroxymethylserine.

35. The method of claim 33 wherein Xaa₂ is glycine, alanine, valine, leucine, isoleucine, threonine, serine, asparagine, methionine, histidine or a-hydroxymethylserine.

36. The method of claim 33 wherein Xaa₃ is lysine.

37. The method of claim 33 wherein Xaa₁ is aspartic acid, glutamic acid, arginine, threonine, or a-hydroxymethylserine, Xaa₂ is glycine, alanine, valine, leucine, isoleucine, threonine, serine, asparagine, methionine, histidine or a-hydroxymethylserine, and Xaa₃ is lysine.

38. The method of claim 37 wherein Xaa₁ is aspartic acid or glutamic acid and Xaa₂ is alanine, glycine, valine, threonine, serine, leucine, or a-hydroxymethylserine.

39. The method of claim 38 wherein Xaa₂ is alanine, threonine, leucine, or α-hydroxymethylserine.

40. The method of claim 39 wherein Xaa₁ is aspartic acid and Xaa₂ is alanine.

41. The method of claim 33 wherein at least one amino acid of P₁ other than β-alanine, when present, is a D-amino acid.

42. The method of claim 41 wherein all of the amino acids of P₁ other than β-alanine, when present, are D-amino acids.

43. The method of claim 33 wherein both P₃peptides are P₁.

44. The method of claim 31 wherein at least one amino acid of P₃ is substituted with (a) a substituent that increases the lipophilicity of the peptide without altering the ability of P₃ to bind copper ions, (b) a substituent that protects the peptide from proteolytic enzymes without altering the ability of P₃ to bind copper ions, or (c) a substituent which is a non-peptide, metal-binding functional group which does not alter the ability of the peptide to bind copper ions.

45. The method of claim 31 wherein P₃ comprises an amino acid sequence which is substituted with a non-peptide, metal-binding functional group to provide the copper-binding capability of P₃.

46. The method of claim 31 wherein L is neutral.

47. The method of claim 31 wherein L is a straight-chain or branched-chain alkane or alkene residue containing from 1-18 carbon atoms.

48. The method of claim 47 wherein L contains 2-8 carbon atoms.

49. The method of claim 31 wherein L is a cyclic alkane residue containing from 2-8 carbon atoms.

50. The method of claim 49 wherein L contains 3-5 carbon atoms.

51. The method of claim 31 wherein L is a nitrogen-containing heterocyclic alkane residue.

52. The method of claim 51 wherein L is a piperazide.

53. The method of claim 31 wherein L is a glyceryl ester.

54. The method of claim 1 wherein the copper chelator is a peptide having attached thereto a non-peptide metal-binding functional group, wherein the peptide comprises a copper-binding site and/or the non-peptide functional group binds copper.

55. The method of claim 1 wherein the animal is in need of the APC because it is suffering from an acquired hypercoagulable state or an acquired protein C deficiency.

56. The method of claim 1 wherein the animal is in need of the APC because it is suffering from sepsis.

57. The method of claim 1 wherein the animal is in need of the APC because it is suffering from a disease or condition involving intravascular coagulation.

58. The method of claim 1 wherein the copper chelator is administered prior to administration of the APC, protein C, activator of protein C or combination of one or more of them.

59. The method of claim 1 wherein the copper chelator is combined with the APC, protein C, agent that increases the synthesis of protein C, activator of protein C, or combination of one or more of them prior to their administration to the animal.

60. A method of treating an animal in need of treatment with activated protein C (APC) comprising:
contacting an effective amount of a copper chelator with a composition comprising one of the following:
(a) APC;
(b) protein C, an agent that increases the synthesis of protein C in the animal, or both;
(c) an activator of protein C; or
(d) a combination of one or more of (a), (b) and (c);
so as to bind any copper present in the composition; and
administering an effective amount of the APC, protein C, protein C, agent that increases the synthesis of protein C, activator of protein C, or combination of one or more of them to an animal in need of treatment with APC.

61. The method of claim 60 wherein the copper chelator is human albumin or a fragment thereof comprising the N-tenninal copper-binding sequence Asp Ala His.

62. The method of claim 60 wherein the copper chelator is a peptide having the formula:
P₁-P₂,
wherein:
P₁ is:
Xaa₁ Xaa₂His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, omithine, phenylalanine, tyrosine, tryptophan, cysteine, methionine, or a-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

63. The method of claim 62 wherein at least one amino acid of P₁ is substituted with (a) a substituent that increases the lipophilicity of the peptide without altering the ability of P₁ to bind copper ions, (b) a substituent that protects the peptide from proteolytic enzymes without altering the ability of P₁ to bind copper ions, or (c) a substituent which is a non-peptide, metal-binding functional group which does not alter the ability of P₁ to bind copper ions.

64. The method of claim 60 wherein the copper chelator is a peptide dimer having the formula:
P₃-L-P₃,
wherein:
each P₃ may be the same or different and is a peptide which is capable of binding copper; and
L is a chemical group which connects the two P₃ peptides through their C-terminal amino acids.

65. The method of claim 64 wherein at least one P₃ is P₁, wherein P₁ is:
Xaa₁ Xaa₂His: or
Xaa₁ Xaa₂His Xaa₃; and
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, p-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or a-hydroxymethylserine; and
Xaa₃ is glycine, alanine, valine, lysine, arginine, ornithine, aspartic acid, glutamic acid, asparagine, glutamine or tryptophan.

66. The method of claim 64 wherein at least one amino acid of P₃ is substituted with (a) a substituent that increases the lipophilicity of the peptide without altering the ability of P₃ to bind copper ions, (b) a substituent that protects the peptide from proteolytic enzymes without altering the ability of P₃ to bind copper ions, or (c) a substituent which is a non-peptide, metal-binding functional group which does not alter the ability of P₃ to bind copper ions.

67. The method of claim 60 wherein the copper chelator is a peptide having attached thereto a non-peptide metal-binding functional group, wherein the peptide comprises a copper-binding site and/or the non-peptide functional group binds copper.

68. The method of claim 60 wherein the copper chelator is removed prior to administration of the APC, protein C, activator of protein C or combination of one or more of them.
